SEQUENCE LISTING (2 sequences)

SEQ ID NO: 1 - DNA, 20 bp, Artificial Sequence
  Synthetic Primer
gagatccctccaaaatcaag

SEQ ID NO: 2 - DNA, 20 bp, Artificial Sequence
  Synthetic Primer
caaagttgtcatggatgacc

(19) United States
(12) Patent Application Publication
(10) Pub. No.: US 2011/0203688 A1
(43) Pub. Date: Aug. 25, 2011

(54) NUCLEIC ACID EXTRACTION ON CURVED GLASS SURFACES

(75) Inventors: Michael W. Reed, Lake Forest Park, WA (US); Oliver Z. Nanassy, Edmonds, WA (US); Paul V. Haydock, Shoreline, WA (US); Daniel P. Gestwick, Seattle, WA (US)

(73) Assignee: BLOOD CELL STORAGE, INC., Seattle, WA (US)

(21) Appl. No.: 12/948,566

(22) Filed: Nov. 17, 2010

Related U.S. Application Data
(63) Continuation of application No. PCT/US2009/063296, filed on Nov. 4, 2009.
(60) Provisional application No. 61/111,079, filed on Nov. 4, 2008.

(51) Int. Cl. F04B 49/00; C07H 21/00; C12P 19/34; C07H 21/04; B65D 71/00

(52) U.S. Cl. 137/565.11; 536/25.41; 435/91.5; 206/223

(57) ABSTRACT

Processes for extracting nucleic acid from a biological sample and related assemblies and kits are disclosed. The processes comprise the steps of (a) providing a device comprising an inner surface, an outer surface, a first port, and a second port, wherein the inner surface is composed of unmodified, smooth glass and defines a tubular lumen providing fluid communication between the first port and second port, wherein the lumen is circular, oval, or elliptical in cross-section, and wherein the lumen is essentially free of nucleic acid-specific binding sites; (b) introducing a nucleic acid-containing sample into the lumen of the device via the first port; (c) allowing nucleic acid in the sample to bind to the unmodified smooth glass surface; and (d) washing the bound nucleic acid.

### CROSS-REFERENCE TO RELATED APPLICATION

This application is a continuation of International Application No. PCT/US2009/063296, filed Nov. 4, 2009, which claims the benefit of U.S. Provisional Application No. 61/111,079, filed Nov. 4, 2008. Each application is incorporated herein by reference in its entirety.

### BACKGROUND OF THE INVENTION

Rapid analysis of nucleic acids from biological samples has been advanced by the development of microfluidic technologies capable of extracting nucleic acids from cell lysates and other sources. Rapid extraction methodologies can be combined with amplification techniques such as polymerase chain reaction (PCR) to provide useful quantities of nucleic acids from minute samples of blood, tissue, cultured cells, or other biological materials. These microfluidic technologies have been widely adopted in biomedical research laboratories, permitting, for example, high-throughput screening of cloned DNA “libraries” from cultured bacteria or other host cells.

Commonly used methods for extracting DNA on such a small scale exploit the tendency for DNA to bind to materials such as silica gel, silica membranes, porous glass, or diatomaceous earth. One such system provides a microcentrifuge tube containing the DNA-binding media (known as a “spin column”). The sample is loaded into the tube and spun in a centrifuge, whereby the DNA is captured and the liquid phase containing contaminants passes through to the bottom of the tube. Such a procedure is disclosed in, for example, U.S. Pat. No. 6,821,757 to Sauer et al. Although spin column technology has been widely adopted by the research community, removal of contaminants is inefficient and the resulting DNA is often of low quality for use in downstream applications such as PCR. Moreover, the need to pipette multiple reagents into open tubes results in a significant risk of sample contamination. Such methods are time consuming when performed manually and very expensive to automate.

The successful use of rapid DNA extraction techniques in research has led to an interest in developing devices and processes through which this technology can be used in medical applications such as point-of-care diagnosis or testing of blood components. Recent progress toward more simple and compact devices has been reviewed by Malic et al., Recent Patents on Engineering 1:71-88, 2007. Despite these recent advances, there remains a need in the art for devices and processes by which high-quality DNA and RNA can be rapidly and economically extracted from biological samples.

### SUMMARY OF THE INVENTION

The present invention provides processes, devices, assemblies, and kits that are useful for the extraction of nucleic acids, including DNA and RNA, from liquid samples.

One aspect of the invention provides a process for extracting nucleic acid from a biological sample. The process comprises the steps of (a) providing a device comprising an inner surface, an outer surface, a first port, and a second port, wherein the inner surface is composed of unmodified, smooth glass and defines a tubular lumen providing fluid communication between the first port and second port, wherein the lumen is circular, oval, or elliptical in cross-section, and wherein the lumen is essentially free of nucleic acid-specific binding sites; (b) introducing a nucleic acid-containing sample into the lumen of the device via one of the first and second ports; (c) allowing nucleic acid in the sample to bind to the unmodified smooth glass surface; and (d) washing the bound nucleic acid to elute contaminants. Within one embodiment, the process further comprises eluting bound nucleic acid from the unmodified smooth glass surface following the washing step. Within other embodiments, the lumen is a linear lumen with a longitudinal axis. Within a related embodiment, at least a portion of the lumen is tapered along the longitudinal axis. Within another embodiment, the lumen is serpentine. Within a related embodiment, the lumen is helical. Within another embodiment, the outer surface comprises a longitudinal ridge. Within an additional embodiment, the device comprises an inner element within the lumen, the inner element comprising an unmodified, smooth glass surface that is convex in cross-section. Within a further embodiment, the process further comprises lysing a cell sample to prepare the nucleic acid-containing sample. Within yet another embodiment, the nucleic acid-containing sample comprises a chaotropic salt. Within additional embodiments, the nucleic acid-containing sample contains animal nucleic acid, human nucleic acid, or microbial nucleic acid. Within another embodiment, the nucleic acid is DNA. Within an additional embodiment, and the nucleic acid is fragmented prior to the introducing step. Within another embodiment, the bound nucleic acid is eluted with a buffer containing a fluorescent compound that exhibits a change in fluorescence intensity in the presence of nucleic acids. Within a further embodiment, flow of liquid through at least a portion of the lumen is turbulent. Within additional embodiments, the process comprises the additional step of amplifying the eluted nucleic acid. The amplifying step may comprise isothermal amplification. Within another embodiment, the washing step comprises introducing a wash reagent into the lumen of the device via said one of the first and second ports, allowing the wash reagent to contact the bound nucleic acid, and removing the wash reagent from the lumen via said one of the first and second ports. Within a further embodiment, the sample is introduced into the lumen and eluted nucleic acid is removed from the lumen via the same port.

Within a second aspect of the invention there is provided an assembly comprising (a) a device comprising an inner surface, an outer surface, a first port, and a second port, wherein the inner surface is composed of unmodified, smooth glass and defines a tubular lumen providing fluid communication between the first port and second port, wherein the lumen is circular, oval, or elliptical in cross-section, and wherein the lumen is essentially free of nucleic acid-specific binding sites; and (b) a pump in fluid communication with the lumen of the device. Within one embodiment, the pump is connected to the second port of the device. Within a related embodiment, the pump is connected to the second port of the device via a manifold. Within a further embodiment, the assembly comprises fluid distribution control means in fluid communication with the pump.

Within a third aspect of the invention there is provided an assembly comprising (a) a plurality of devices, wherein each device comprises an inner surface, an outer surface, a first port, and a second port, wherein the inner surface is composed of unmodified, smooth glass and defines a tubular lumen providing fluid communication between the first port and second port, wherein the lumen is circular, oval, or elliptical in cross-section, and wherein the lumen is essentially free of nucleic acid-specific binding sites; (b) a manifold comprising a plurality of connectors, each connector adapted to receive one of the devices and provide a fluid pathway into the lumen thereof via one of the ports; and (c) a pump in fluid communication with the manifold, wherein each of the plurality of devices is coupled to a connector of the manifold.

Within a fourth aspect of the invention there is provided a kit comprising (a) a device comprising an inner surface, an outer surface, a first port, and a second port, wherein the inner surface is composed of unmodified, smooth glass and defines a tubular lumen providing fluid communication between the first port and second port, wherein the lumen is circular, oval, or elliptical in cross-section, and wherein the lumen is essentially free of nucleic acid-specific binding sites; and (b) a buffer in a sealed container. The buffer may be a lysis buffer, a wash buffer, or an elution buffer. Within one embodiment, the buffer is an elution buffer. Within a related embodiment, the buffer is an elution buffer that comprises a fluorescent compound that exhibits a change in fluorescence intensity in the presence of nucleic acids, such as a bis-benzimidine compound.

These and other aspects of the invention will become evident upon reference to the following detailed description of the invention and the attached drawings.

All references cited herein are incorporated by reference in their entirety. Numeric ranges recited herein include the endpoints.

### BRIEF DESCRIPTION OF THE DRAWINGS

FIG. 1 illustrates an arrangement comprising a nucleic acid extraction device and a pump.

FIG. 2 illustrates an arrangement comprising a plurality of nucleic acid extraction devices, a manifold, and a pump.

FIG. 3 illustrates an Archimedean spiral.

FIG. 4 illustrates a Fermat's spiral.

FIG. 5 illustrates the results of amplification of DNA recovered from a curved glass surface.

FIGS. 6A and 6B illustrates a portion of a nucleic acid extraction device.

FIGS. 7A and 7B illustrate a portion of a nucleic acid extraction device.

FIGS. 8A and 8B illustrate a nucleic acid extraction device comprising an end cap.

### DESCRIPTION OF THE INVENTION

The present invention provides for the extraction of nucleic acids, including deoxyribonucleic acids (DNA) and ribonucleic acids (RNA), from biological samples. As used herein, the term “biological sample” means a sample containing cells or cell components and includes any sample, liquid or solid, that contains nucleic acids. Suitable biological samples that can be used within the invention include, without limitation, cell cultures, culture broths, cell suspensions, tissue samples, cell lysates, cleared cell lysates, whole blood, serum, buffy coat, urine, feces, cerebrospinal fluid, semen, saliva, wound exudate, viruses, mitochondria, and chloroplasts. In one embodiment, the sample is blood or a blood product (e.g., platelets) and the nucleic acids that are extracted are those from contaminant bacterial pathogens in the blood or blood product.

DNA produced through the present invention has been found to be of high quality for downstream applications (e.g., amplification). In comparison to porous glass surfaces, the smooth glass surfaces used in the invention are easy to wash free of enzymes, metals (e.g., heme), and other protein contaminants that can interfere with PCR-based assays. PCR yields were improved and variability decreased. The devices of the invention also allow the extracted nucleic acids to be concentrated. For example, DNA captured from a 0.5-mL sample can be concentrated in 0.1 mL of elution buffer by sweeping the buffer through the lumen of the device. This concentration effect is valuable for dilute samples or pathogen detection with improved sensitivity.

In contrast to the spin columns that are currently in widespread use, the present invention incorporates nucleic acid extraction devices that can be closed off from the outside environment. The invention thus provides systems in which the contents of the extraction device are essentially isolated from the environment, although these systems comprise provisions (e.g., sealable ports or fittings) that allow for introduction of samples and reagents, and removal of waste products, washes, and extracted nucleic acids. For many applications such closed systems are preferred because they are inherently resistant to contamination.

Devices used within the present invention have significantly lower surface area:volume ratios than known devices employing porous silica substrates, yet efficiently extract DNA from liquid samples. Porous silica substrates in cylindrical devices such as spin columns have a glass surface area of hundreds of mm² per μL of void volume. For example, a 0.6 mm×5 mm diameter cylinder packed with 10-μm porous silica beads will have a glass area of approximately 3684 mm² and a void volume of 5.641 μL, resulting in a surface area:void volume ratio of 653 mm²/μL. In contrast, devices of the present invention have surface area:void volume ratios of from 0.1 mm²/μL to 20 mm²/μL, more commonly from 0.25 mm²/μL to 10 mm²/μL, and usually from 0.5 mm²/μL to 5 mm²/μL. Typical Pasteur pipettes, which can be used within the invention, have surface area:volume ratios of about 0.57 mm²/μL in the larger end and 4 mm²/μL in the smaller end.

Nucleic acid extraction devices used within the present invention comprise first and second ports through which a nucleic acid-containing sample can be introduced, and through which contaminants and the extracted nucleic acid can be removed. The devices further comprises a tubular lumen defined by the inner surface of the device, wherein the inner surface is composed of unmodified, smooth glass. The lumen, which is circular, oval, or elliptical in cross-section, is essentially free of nucleic acid-specific binding sites and is in fluid communication with the two ports. Within the practice of the invention, nucleic acids are bound to the inner surface of the device. In addition, the device is designed to enable a bolus of liquid to move through the device without an air bubble penetrating the leading edge and becoming entrained in the bolus. The device can be sized to optimize performance with different types of samples. Parameters to be considered in optimizing performance include the diameter and length of the lumen. For example, the volume of the lumen can be selected based on the volume of the sample. A wider diameter lumen may improve flow rate with more viscous samples.

Those skilled in the art will recognize that, in view of the fabrication methods involved, the inner surface of the device may exhibit irregularities in shape. Such irregularities may arise, for example, as artifacts of the fabrication process (e.g., tolerance variations). It is generally desirable to minimize such irregularities to the extent practicable.

In one embodiment of the invention the lumen is a linear lumen. Within this embodiment, the device will commonly comprise a straight tube with a central lumen. The diameter of the lumen can be essentially constant throughout its length. In the alternative, the lumen can be tapered along its longitudinal axis. The entire lumen can be tapered, or the taper restricted to a small section of the lumen. A device exemplifying the latter arrangement is a Pasteur pipette.

In other embodiments of the invention the lumen is curved along its central axis. A variety of curved conformations are contemplated. Representative curved lumens include, without limitation, those having a C or S shape, and more extensive serpentine lumens comprising a plurality of bends, spirals, and helical coils. A high ratio of lumen volume to overall device volume can be obtained by curving the lumen through three dimensions. The invention thus includes lumens comprising, for example, a plurality of serpentine channels arrayed in parallel planes or a plurality of coaxial helical channels. Devices of this type are conveniently constructed from readily available forms of glass tubing, such as capillaries, gas chromatography columns, condenser tubes, and the like.

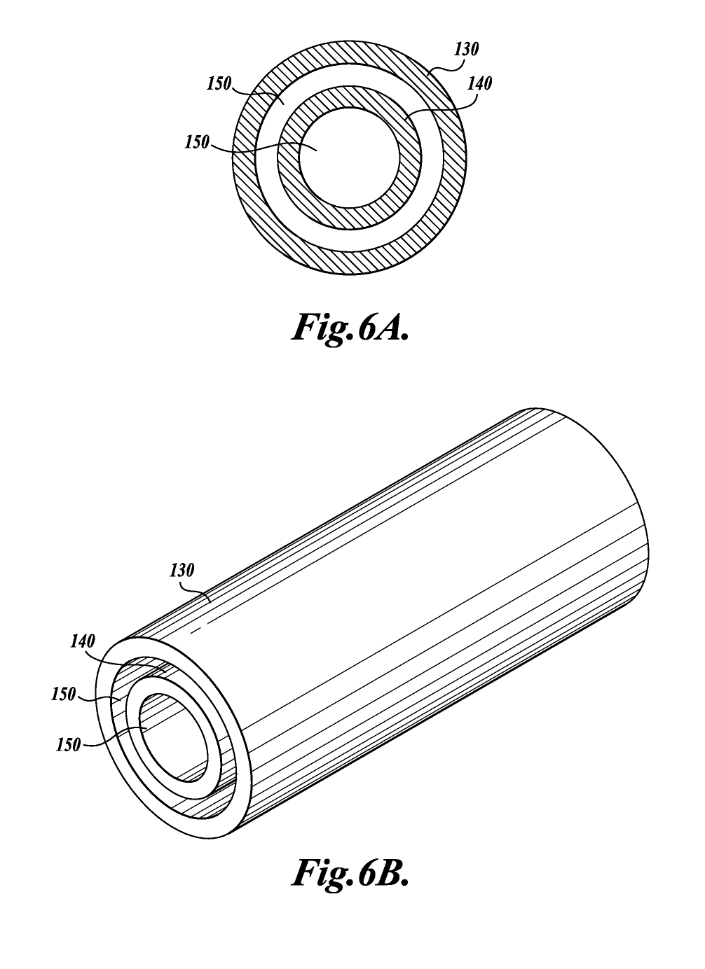

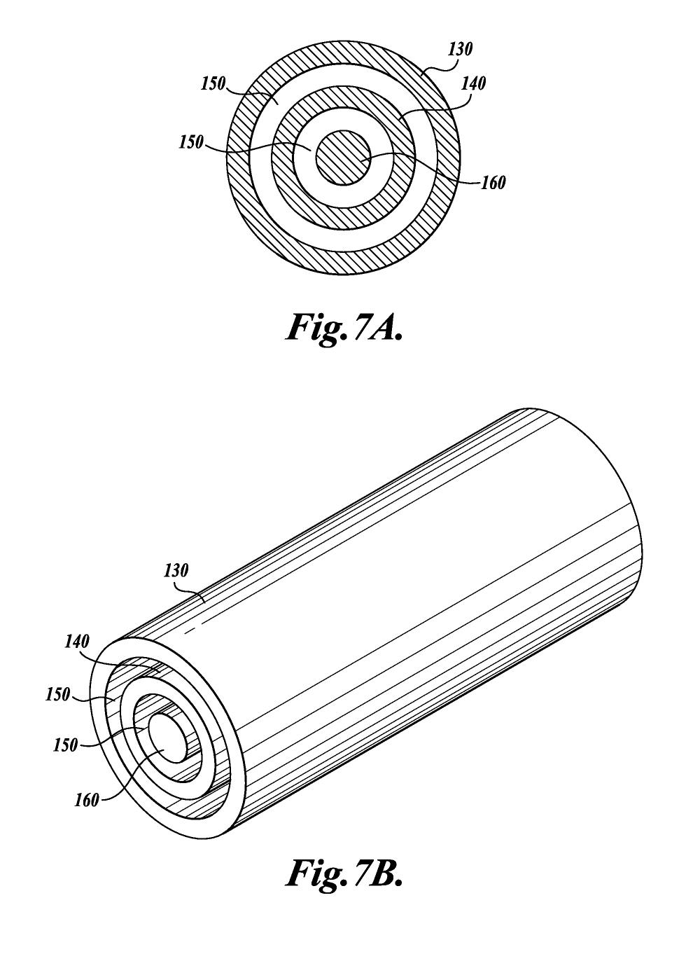

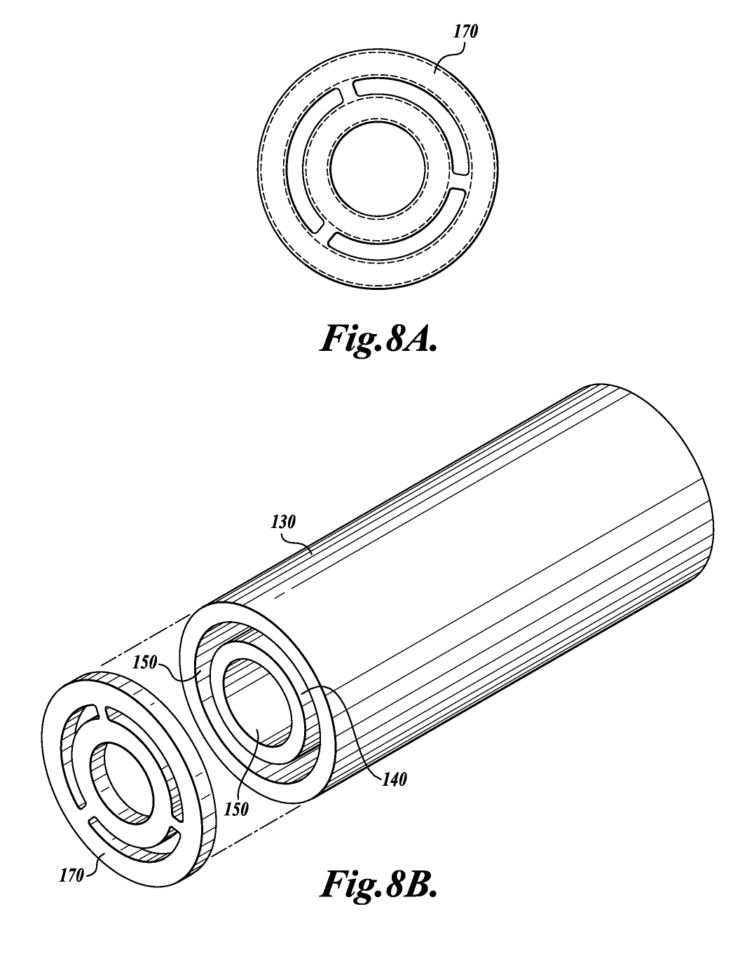

In a basic embodiment, the device consists of an inner surface, an outer surface, a first port, and a second port, the inner surface defining the lumen that provides fluid communication between the first port and second port. In other embodiments the device comprises an inner element within the lumen, the inner element comprising an unmodified, smooth glass surface that is convex in cross-section. Such devices can comprise a plurality of essentially concentric binding elements, such as tubes or rods, thereby providing a plurality of unmodified, smooth glass binding surfaces in the lumen of the device. FIGS. 6A and 6B illustrate examples of such devices in which concentric glass tubes 130 and 140 define two lumens 150. The outer lumen has both concave and convex walls, while the inner lumen has a concave wall. FIGS. 7A and 7B illustrate another embodiment that comprises, in addition to concentric tubes 130 and 140, a central glass rod 160. Within this embodiment, both inner and outer lumens 150 have both a concave and a convex wall. Such configurations of tubes and/or rods can be stabilized through the use of retention elements as disclosed below. As shown in FIGS. 8A and 8B, this arrangement can be further stabilized by providing an end cap 170 distal to the retention element. The retention element and end cap will be configured to allow fluid flow therethrough to all glass surfaces within the device.

When glass tubes are utilized within the present invention, the ends of the tube can provide the inlet and outlet ports, with the intermediate portion defining the lumen. The ends of the tube (inlet and outlet ports) can be fitted with endcaps or other fittings through which reagents are added and withdrawn, as disclosed in more detail below. Such fittings can also seal the device. Such devices can further comprise a protective housing, guard, handle, or the like to facilitate handling and protect the tube from breakage. These elements are conveniently constructed from polymeric materials. Those skilled in the art will recognize that a glass tube can be fitted to a housing whereby inlet and outlet ports are formed as openings through the surface of the housing to provide fluid access to the glass tube.

In one embodiment, the shape and proportions of at least a portion of the lumen are selected to provide for turbulent flow of liquids passing therethrough. Turbulent flow can facilitate the mixing of liquids passing through the lumen. Whether flow is turbulent or laminar can be characterized by its Reynolds number (Re). The Reynolds number can be described as the ratio of inertial forces over viscous forces, where viscous forces can be thought of as a resistance to velocity and inertial forces can be thought of as a resistance to change in velocity.

Re=(p×Vs×L)/(u), where:

p=fluid density (kg/m3)

Vs=mean fluid velocity (m/s)

L=characteristic length (m), which for pipes is Dh=hydraulic diameter (m)

Dh=(4×Area)/(perimeter), i.e., area and perimeter of pipe cross section.

u=absolute viscosity (s N/m2)

When Re is below 2300, the flow is considered laminar, and when Re is above 4000 the flow is considered turbulent. Anything between these two values is considered a transition region.

A typical Pasteur pipette is of varying diameter, having two uniform diameter sections at either end connected by a tapered portion. For simplicity, the Reynolds number in the two uniform diameter sections is calculated below. The narrow section has a diameter of 0.9 mm, and the larger section has a diameter of 5 mm. Flow rates will generally not exceed 600 μL/second, and will typically be approximately 60 μL/second. Using the above equation and the values:

L=0.0009 m (small section) or 0.005 m (large section)

Vs=0.94 m/s (small section, high flow); 0.094 m/s (small section, low flow); 0.03 m/s (large section, high flow); and 0.003 m/s (large section, low flow)

p(water)=1000 kg/m3

u(water)=1/1000 sN/m2

Re=1000×0.094×0.0009×1000=84.9, at a flow rate 60 μL/second in the small section; and Re=1000×0.003×0.005×1000=15.3, at a flow rate of 60 μL/second in the a large section. At a flow rate of 600 μL/second, Re=849 in the small section and Re=153 in the large section. Thus, devices having the above-disclosed dimensions can accommodate flow rates in excess of 1625 μL/second before Re approaches the transition region.

Within one embodiment of the invention, the lumen is serpentine in shape. As used herein, “serpentine” lumens include planar lumens that bend in two dimensions as well as three-dimensional pathways having the form of a helix and variants thereof. Such three-dimensional structures can be circumferentially flattened along at least one side to reduce overall device volume. A serpentine shape allows for exposure of the sample to a large surface area of glass, while keeping the cross-section of the lumen and the overall device small. Limiting the lumen cross-section dimensions contributes to the prevention of air bubbles slipping past the leading edge of a liquid bolus within the lumen. The serpentine design also allows this combination of high surface area (glass-liquid interface) and small cross-section to exist within a compact footprint. As discussed above, serpentine (including helical) lumens include those with circular cross-sections and other configurations.

Devices of the present invention comprise an inner surface composed of unmodified, smooth glass. This surface is effective for binding nucleic acids, including DNA and RNA. As used herein, an “unmodified smooth glass surface” means a glass surface having a smoothness corresponding to that of a standard microscope slide, Pasteur pipette, glass capillary, or the like, wherein the surface has not been etched or otherwise altered to increase its surface area, and wherein it has not been modified to specifically bind nucleic acids as disclosed below. Specifically excluded from “smooth glass” is porous glass that is known in the art to capture nucleic acids, commonly in bead, frit, or membrane form. Such porous glass commonly has pores sized within the range of 0.1 μm to 300 μm. Suitable glass materials for use within the present invention include soda lime glass (e.g., Erie Electroverre Glass; Erie Scientific Company, Portsmouth, N.H.), boro silicate glass (e.g., Corning 0211, PYREX 7740; Corning Incorporated, Corning, N.Y.), zinc titania glass (Corning), and silica glass (e.g., VYCOR 7913; Corning Incorporated). Suitable for use within the invention is glass tubing, which is readily available in a variety of sizes. Of particular interest are Pasteur pipettes, which are inexpensive, provide a good surface:volume ration, and include a large diameter region within the lumen to facilitate mixing of reagents. As discussed above, glass capillaries, chromatography columns, condenser tubes, syringes, rods, and the like having smooth glass surfaces can also be employed. The lumen is essentially free of nucleic acid-specific binding sites, such as charged surfaces or binding sites provided by immobilized oligonucleotides, minor groove binding agents, intercalating agents, or the like. A lumen that is “essentially free of nucleic acid-specific binding sites” is one that does not contain an amount of such sites sufficient to give a statistically significant increase in nucleic acid binding as compared to glass.

In its simplest form, the device used within the invention is a glass tube with a port at each end. Those skilled in the art will recognize that other configurations can be employed, and that glass tubes of various shapes can be incorporated into larger and more complex devices. These other devices can be configured to, for example, facilitate automated handling, increase durability by protecting fragile glass elements, or connect to other devices used for upstream or downstream handling of samples. The remainder of the body of such a device is preferably made from materials that exhibit low auto-fluorescence and very low binding of nucleic acids. The materials should also be impervious to reagents with which they may come into contact during use (e.g., ethanol). Rigid or semi-rigid, organic polymeric materials are preferred. Representative such materials include acrylic (a high molecular weight rigid material), polycarbonate, polypropylene (a low surface energy thin film), cellulose acetate, polyethylene terephthalate (PET), polyvinylchloride, and high density polyethylene (HDPE), but not polystyrene. Suitable adhesive materials for bonding polymeric materials include, without limitation, 300LSE adhesive film (3M); 467 acrylic adhesive film (3M Company, St. Paul, Minn.); 8141 acrylic adhesive film (3M Company); and Transil silicone adhesive film. Outgassing of certain adhesives after device manufacture may reduce DNA yield; vacuum degassing can be used to alleviate this issue.

The device further comprises ports through which liquids can be introduced into or removed from the lumen. Thus, the ports provide openings through the surface of the device and are in fluid communication with the lumen. In the simplest configuration, the inlet and outlet ports are provided as openings in the device, such as openings at tube ends. Such openings are conveniently circular in shape, although shape is a matter of routine design choice. Devices in which the ports are provided by the ends of glass tubing can be inserted directly into a manifold or other retention element as disclosed in more detail below. The inlet and outlet ports can further comprise additional components, allowing the sample and other reagents to be introduced into the device by various means. For example, Peek tubing stubs can be attached to the device to allow manual input. Manual addition allows the various buffers to be optimized for volume, incubation time, and flow rate. In the alternative, standard 1-ml polypropylene syringes or a programmable peristaltic pump can be used with tubing and Luer-lock adaptors. Within another embodiment, the inlet and outlet ports are provided by small diameter holes sized to accept a needle (e.g., a blunt tip, 22G needle) inserted into the hole. Connections to the needles are made using Luer-lock fittings. In another embodiment, each of the inlet and outlet ports comprises an elastomeric septum or cap that can be pierced with a needle or cannula, thus providing a device that is sealed until the time of use. Ports can be further sealed against leaks by the inclusion of O-rings, gaskets, or the like.

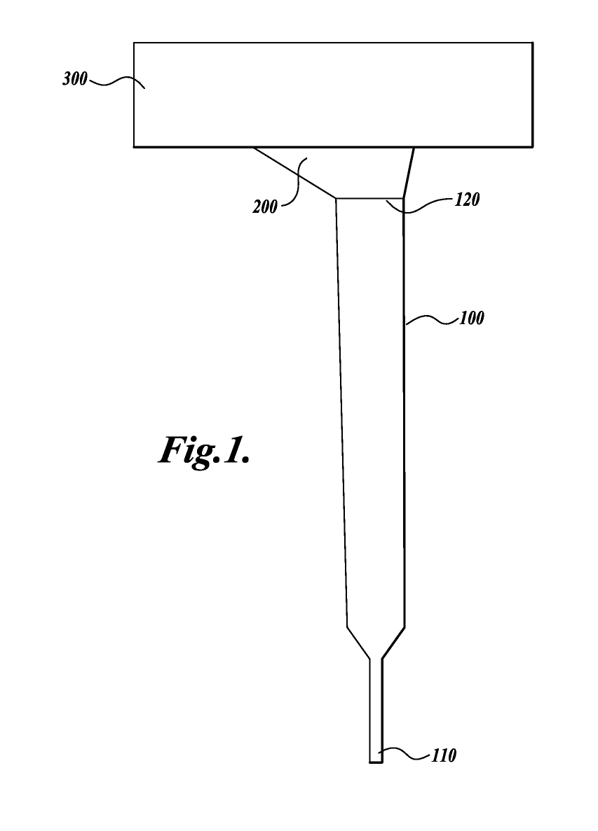

FIG. 1 illustrates an assembly of the invention comprising device 100 and pump 300. Second port 120 of device 100 is inserted into retention element 200. Retention element 200 is constructed by known methods, such as injection molding. Retention element 200 is coupled to pump 300 and provides for fluid communication with the lumen of device 100. In this arrangement, pump 300 can apply suction and draw liquids into device 100 via first port 110. In the alternative, liquids can be delivered into the lumen of device 100 via second port 120. In the illustrated embodiment, retention element 200 is designed to retain device 100 in a stable position relative to pump 300. Those skilled in the art will recognize that retention element 200 can be configured in a variety of alternative ways. For example, retention element 200 can be constructed from flexible or rigid tubing, and device 100 can be held in a fixed position using a clamp or the like. In an illustrative example, 0.25″ i.d. polyurethane (e.g., TYGON) tubing forms a tight seal with a conventional Pasteur pipette having a 0.27″ o.d. larger end. This size tubing also tightly mates with the tip of a 1-ml syringe or a hand-held pipettor. Such retention elements are readily prepared using thin-wall (e.g., 1/32 inch) tubing cut in ⅜ inch lengths.

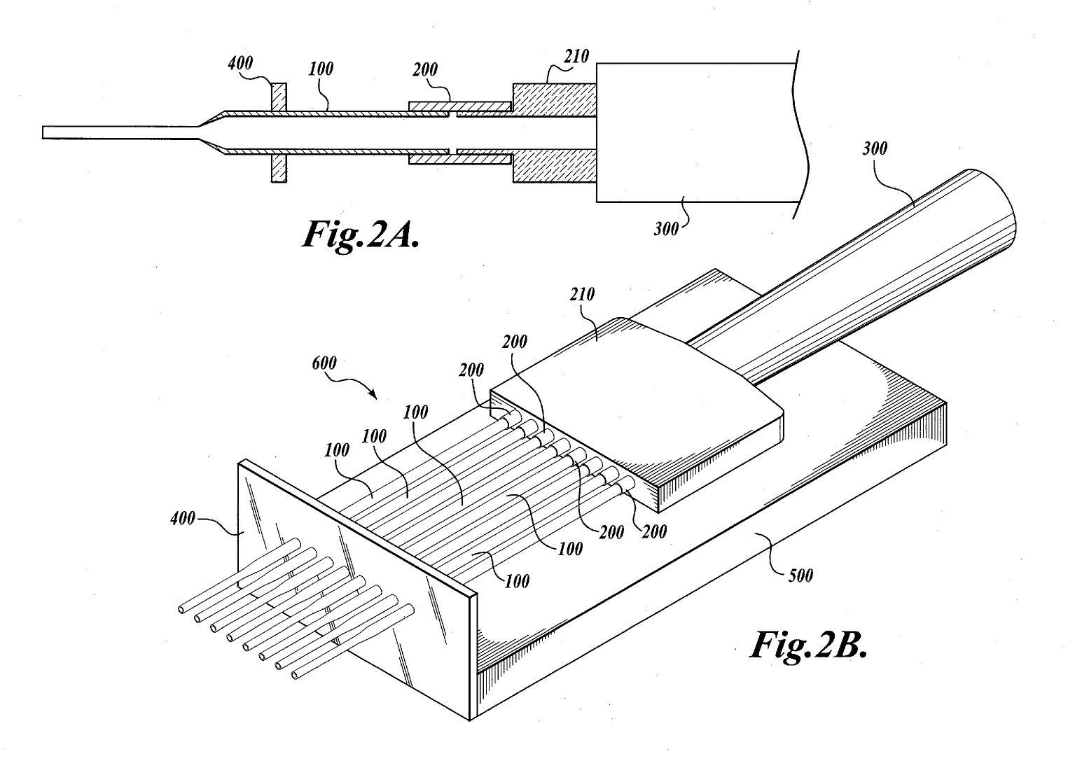

The arrangement of FIG. 1 is readily modified as shown in FIGS. 2A and 2B to provide for simultaneous use of a plurality of devices 100. In this latter arrangement, shown as assembly 600, devices 100 are connected to manifold 210 via retention elements 200, which are constructed from thin-wall polyurethane tubing. Manifold 210 is in turn coupled to pump 300 and provides fluid communication between pump 300 and devices 100. Such multi-device assemblies can be configured so that the plurality of devices 100 are positioned to correspond to wells of standard multi-well plates, such as 96-well plates. In the illustrated assembly, eight devices 100 are held in position by alignment plate 400 to align with a row of eight wells in a 96-well plate. In such an arrangement, the assembly can draw fluids from and expel fluids into one or a series of such plates. Samples and reagents (e.g., wash and elution buffers) can be arrayed in different rows of a single plate, and either the plate or the assembly is moved to insert the ends of the devices into the appropriate wells. This process can be carried out manually or automated. Multi-well plates are available in a range of well volumes (e.g., 200 μL, 0.5 mL, 1.0 mL, 2.0 mL) to provide a flexible system and facilitate concentration of nucleic acids from dilute samples. As will be recognized by those of ordinary skill in the art, other vessels, such as tubes (e.g., microcentrifuge tubes), plates, or dishes can also be used. Tubes can be arranged in a multi-well plate format. When glass tubes are used, the interior of the tube provides a further smooth glass surface that can be used for nucleic acid capture. In this arrangement, nucleic acid eluted from the glass surfaces of the device and the tube can be collected in the device and transferred to another vessel, or can be collected in the tube. For such multi-device assemblies, each device in the assembly can be run individually, or all devices in the assembly can be run simultaneously.

FIG. 2B shows an assembly further comprising a handling plate 500 to which the remainder of the assembly is fixed. Handling plate 500 further stabilizes the components of assembly 600 and allows three-dimensional rotation of the entire assembly. In a typical nucleic acid extraction procedure, a nucleic acid-containing sample in binding/lysis buffer is drawn into devices 100 by pump 300, and nucleic acid is allowed to bind to the inner walls of the devices. With the liquid in the devices, assembly 600 is optionally tipped to the side and rotated to maximize contact between sample and glass in the upper (wide) section of devices 100. The liquid is then expelled, and a first wash buffer is drawn into the devices. The buffer is pumped up and down within the lumens of the devices by the action of pump 300. The buffer is then expelled, and the wash is repeated as required. After the final wash, a stream of air is passed through devices 100 to dry bound nucleic acid. Depending on the type of pump 300, air drying may be facilitated by disconnecting devices 100 from pump 300 (with or without manifold 210) and connecting them to an air stream provided by other means. Finally, the nucleic acid is eluted from devices 100 and transferred into a 96-well plate, a set of tubes, or the like. Pump 300 can also be used to pre-wash or pre-treat the interior surfaces of devices 100.

Additional automation can be provided by connecting these assemblies to a valve mechanism connected to a microprocessor-controlled, multi-channel pump and fluid distribution control means as disclosed in more detail below. Such assemblies can be combined with standard laboratory robotic systems to provide for fully automated sample handling.

The device will commonly take the form of a length of tubing, wherein the outer cross-section is the same shape as the cross-section of the lumen. This form of the device is inexpensive, easy to store and handle, and provides considerable flexibility in use.

Within one embodiment, the outer surface of the device comprises at least one longitudinal ridge. A ridged device can be used to disrupt tissue during sample collection and/or mix samples prior to introduction into the lumen of the device. In a typical application, a nucleic-acid containing material is placed in a tube with buffer, the ridged device is inserted into the tube and spun to mix the sample, and the sample is drawn into the device.

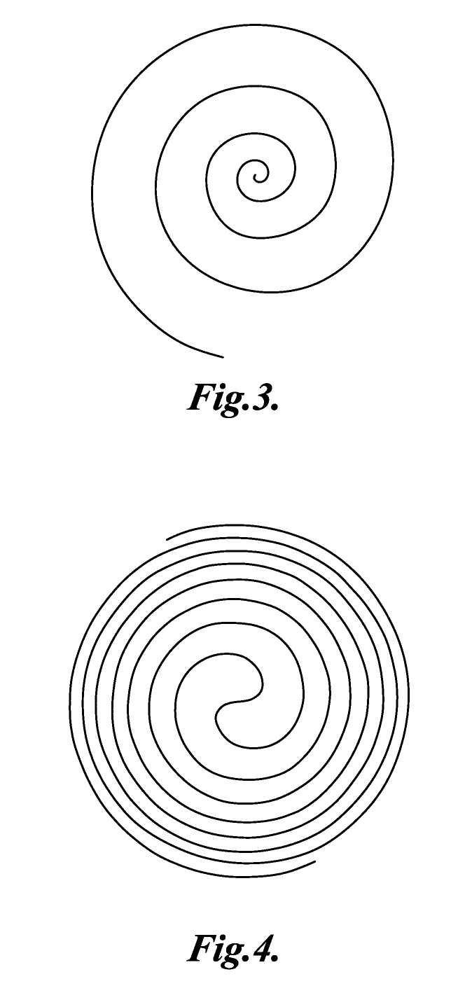

In another embodiment, a tubular device as disclosed above is contained within a larger structure as disclosed briefly supra. Such an arrangement is particularly advantageous when using a device with a serpentine lumen to protect the glass from breakage and facilitate handling. For example, a spiral-shaped capillary tube can be enclosed within a card-like or block-like body prepared from adhesive, resin, epoxy, or the like. The term “spiral” is used herein for its ordinary meaning, that is a planar curve winding in a continuous and gradually widening form about a central point. Examples of suitable spirals include Archimedean spirals (FIG. 3) and Fermat's spirals (FIG. 4), although other shapes can be employed. See, for example, Wikipedia (en.wikipedia.org/wiki/Spiral). Glass tubing (e.g., capillary tubes) can be bent into the desired spiral shape by heating a straight glass capillary tube to its softening point and winding it onto a reel with sidewalls designed to keep the tube aligned. The spiral can be constructed as a single-plane structure or in multiple planes (i.e., two or more spirals sitting flat on top of each other). The ends of the spiral are bent to face and protrude upwards slightly from the plane of the spiral to provide the first and second ports. The ends are then covered, and the body material (e.g., adhesive, resin, or epoxy) is poured or sprayed onto the spiral to provide strength and ease of handling. A mold can be used to create the desired shape, which may include alignment holes, slots, or protrusions to facilitate mating the device to a holder or manifold. After the material has hardened, the tube ends (ports) are uncovered. In a typical embodiment, the resulting structure is in the form of a flat disc with first and second ports on its upper surface. The ports can be provided with additional components as disclosed in more detail supra. A viewing window may be provided by leaving a hole in the body material.

Alternative methods of construction will be evident to those of ordinary skill in the art. For example, laminated plastic construction can be employed essentially as disclosed by Reed et al., U.S. 20090215125 A1. Briefly, individual polymeric layers are cut to shape using known methods such as laser cutting, CNC drag knife cutting, and die cutting. Adhesive layers are prepared to go between the layers of dry plastic. The adhesive layer will ordinarily be a pressure-sensitive adhesive available in a thin film that can be cut using the same method used for the plastic. Adhesives may be used in an Adhesive-Carrier-Adhesive (ACA) format where the carrier is preferred to be the same material as used in the other layers of the device. Other methods of applying liquid adhesives, such as screen printing, may also be employed. The several layers are registered to each other and pressed together. Features to assist in registration, such as alignment holes, are advantageously incorporated into the final design. Pressure and temperature during the cure cycle are adhesive-dependent; selection of suitable conditions is within the level of ordinary skill in the art. In the alternative, the device can be assembled through the use of a compression seal as disclosed in 20090215125 A1. Lamination can incorporate molded elements as disclosed supra.

The invention also provides an assembly comprising a device as disclosed herein and a pump in fluid communication with the lumen of the device. The term “pump” is used herein to include both manually operated (e.g., syringes and multi-channel pipettors) and powered (e.g., electric) devices. The assembly is configured so that the pump can deliver fluids into the lumen and remove them from the lumen via one or both of the ports. The pump is selected for its ability to meet the following criteria: (1) ability to accurately dispense volumes in the range of 20 μL to at least 1000 μL, and preferably up to 2.5 mL; (2) ability to effectively pump air as well as liquids; and (3) ability to operate in reverse. Syringe-type or bellows-type pumps satisfy these criteria and allow the device to be operated in the manner of a conventional pipette, wherein one of the first and second ports is used for the introduction and removal of all reagents. When liquids are moved through both ports, it is advantageous to use a pump that also provides a low or zero dead volume to minimize cross contamination of reagents and has wetted surfaces made of materials compatible with the various reagents used (e.g., chaotropic salts and ethanol). Peristaltic pumps offer a good working combination of all of these traits, but do not offer the most accurate volume dispensing of all pump options. Peristaltic pumps are advantageously used when larger volumes of liquids are handled. Computer-controlled multi-channel peristaltic pumps (e.g., ISMATEC 12-channel pumps; Ismatec SA, Glattbrugg, Switzerland) will accommodate multiple devices simultaneously and can be programmed to start/stop/change flow rate or reverse direction of flow. When employing other pump styles, multiple pumps may be required for particular functions, although such an arrangement will complicate the overall fluid management system.

The assemblies of the present invention may further include fluid distribution control means in fluid communication with the pump. The fluid distribution control means comprises one or more valves that allow for a plurality of fluids to be sequentially pumped through the device, typically in the form of a valve-manifold block. It is preferred that manifold inputs and the exit pass through sterile filters to protect the valve-manifold assembly from contamination, and that the exit line have a check valve to prevent backflow from the pump tubing into the manifold. An exemplary fluid distribution control means is a model V-1241-DC six-position, seven-port rotary selector valve manufactured by Upchurch Scientific, Oak Harbor, Wash. This selector valve allows the introduction of air gaps between reagents. The fluid distribution control means may further comprise a programmable computer, either external to the valve mechanism or fully integrated therewith. In certain embodiments of the invention, the programmable computer is a desktop or laptop personal computer. In other embodiments, the programmable computer is a dedicated microprocessor device. In an exemplary system, control of fluid distribution is achieved using the above-disclosed selector valve in combination with a multi-channel peristaltic pump using an application written in Visual Basic for Microsoft Excel and running on a personal computer. Both the valve mechanism and the pump feature RS232 control interfaces. These components are addressed using Excel through the USB port of the computer and a USB-to-Serial converter. As will be understood by those skilled in the art, custom firmware software may also be employed.

Liquid reagents are conveniently stored in septum-sealed vials equipped with a sterile filter vent. The vials may be connected to the fluid distribution control means using a standard Luer-type needle inserted through the septum and connected to manifold inputs via microbore tubing.

After fabrication, the device is preferably treated with ethylene oxide or gamma sterilization to remove pathogens. Reagents for use with the device preferably pass a 2-micron cellulose filter on entry to remove contaminants. Other methods of removing contaminants, including contaminants that may interfere with nucleic acid amplification, are disclosed by Reed et al., WO 2008002882. The reagent ports on the device may provide an interface to yellow and blue pipette tips. A needle-septum interface can be provided.

Liquid samples are ordinarily introduced into the device at flow rate of approximately 0.1 mL/minute to approximately 5.0 mL/minute, although, as disclosed above, considerably higher flow rates can be used. The actual flow rate is design-dependent, taking into consideration the total volume of the fluid pathway and the configuration of the lumen.

Dilute or concentrated samples can be prepared for input into the device. Lysis and digestion of intact cells releases DNA or RNA from residual proteins (for example histones). In the alternative, solid samples (e.g., bacterial spores or dried blood on cloth) or semisolid samples (e.g., mouse tails or sputum/stool) can be homogenized and lysed before input to the device to provide a homogeneous and non-viscous sample that will flow through the lumen of the device. More viscous samples, such as blood, can also be used.

Nucleic acids are bound to the glass surface(s) of the device in the presence of a salt (e.g., KCl) at a concentration of at least 0.5 M to about 2 M or more depending on solubility, or a chaotrope (e.g., guanidine HCl or guanidine thiocyanate) at a concentration of at least 1 M to about 6 M or the limit of solubility. Binding of nucleic acids is ordinarily done at a pH of approximately 5 to 8, preferably about 6. The lumen is then washed using buffered solutions of decreasing salt concentration. As salt concentration decreases, ethanol is added to the wash solution to retain the nucleic acid on the glass and to remove contaminants that may interfere with downstream processes such as nucleic acid amplification. Washing is carried out at pH 6-9, commonly pH 6-8. Nucleic acids are eluted from the device with a low-salt solution at basic pH, commonly pH 8-9.

In general, when cells are present within the biological sample they are lysed to provide a cell lysate from which the nucleic acids are extracted. A variety of methods of cell lysis are known in the art and are suitable for use within the invention. Examples of cell lysis methods include enzymatic treatment (using, for example, proteinase K, pronase, or subtilisin), mechanical disruption (e.g., by sonication, application of high pressure, use of a piezobuzzer device, or bead beating), or chemical treatment. Beads used for mechanical disruption should be made of a substance that does not bind nucleic acids under the disruption conditions. Suitable substances include acrylic, polycarbonate, polypropylene, cellulose acetate, polyethylene terephthalate, polyvinylchloride, and high density polyethylene. Lysing cells in the sample by treating them with a chaotropic salt solution is particularly advantageous. Methods and reagents for lysing cells using chaotropic salts are known in the art, and reagents can be purchased from commercial suppliers. Specific reagent compositions and reaction conditions will be determined in part by the type of cell to be lysed, and such determination is within the level of ordinary skill in the art. Suitable chaotropic salts include guanidinium thiocyanate, guanidine hydrochloride, sodium iodide, and sodium perchlorate. Guanidine hydrochloride, which is preferred for lysing blood cells, is used at concentrations of 1M to 10M, commonly 1M to 5M, usually 1M to 3M. Higher concentrations of sodium iodide are required, approaching the saturation point of the salt (12M). Sodium perchlorate can be used at intermediate concentrations, commonly around 5M. Neutral salts such as potassium chloride and sodium acetate can also be used to obtain binding of nucleic acids to glass surfaces, and may be used in place of chaotropic salts when cell lysis is not required or is achieved by other means (e.g., in the case of bacterial cell lysis). When using neutral salts, the ionic strength of the buffer should be at least 0.25M. An exemplary lysis buffer is a 2M solution of guanidinium thiocyanate (GuSCN) buffer at pH 6.4. Lysis in a chaotropic salt solution also removes histone proteins from genomic DNA and inactivates nucleases. Lysis buffers will generally also contain one or more buffering agents to maintain a near-neutral to slightly acidic pH. A suitable buffering agent is sodium citrate. One or more detergents may also be included. Suitable detergents include, for example, polyoxyethylenesorbitan monolaurate (TWEEN 20), t-octylphenoxypolyethoxyethanol (TRITON X-100), sodium dodecyl sulfate (SDS), NP-40, CTAB, CHAPS, and sarkosyl. Alcohol, commonly ethanol, is included in the lysis and wash solutions, with the actual concentration selected to compensate for the lowered salt concentration in the washes. In the absence of salt, alcohol is included at a concentration of at least 50%, with 70% alcohol preferred in the final wash. If salt is included in the reagents, alcohol concentration will ordinarily range between 10% and 80%, often between 10% and 60%, usually between 20% and 50%. Optimization of buffers is within the level of ordinary skill in the art. Lysis is generally carried out between room temperature and about 95° C., depending on the cell type. Blood cells are conveniently lysed at room temperature. It is generally preferred that the use of silica particles in cell lysis be avoided, since silica particles may bind nucleic acids and reduce the efficiency of the extraction process. Although not necessary, DNA may be sheared prior to loading the lysate into the extraction device. Methods for shearing DNA are known in the art.

The nucleic acid-containing sample is introduced into the device via one of the ports. Nucleic acid is captured on the glass surface(s) in the presence of a salt or chaotropic salt as disclosed above. Satisfactory binding of nucleic acids to glass is achieved at room temperature (15°-30° C., commonly about 20° C.), although the extraction process can be run at higher temperatures, such as up to 37-42° C. or up to 56° C., although higher temperatures may reduce recovery of nucleic acids. The sample may be allowed to stand in the device for a period of time, and the sample solution may be pumped back and forth through the lumen. Wash buffers are then pumped into one port, such as by use of a peristaltic pump, a syringe, or a pipetter. Selection of wash buffers will depend in part on the composition of the sample loading solution. In general, salt concentration will be reduced during the washing process, and pH will be increased slightly. If the lysis buffer contains a chaotropic salt, the initial wash will commonly also contain that salt at the same or somewhat lower concentration (e.g., 1-3M GuSCN). The final wash should reduce the ethanol concentration to below 50%, preferably to about 10%-20%, to minimize inhibition of nucleic acid amplification in downstream processing. The alcohol content of wash solutions will ordinarily range between 20% and 80%. Wash solutions containing at least 50% ethanol, preferably about 70% ethanol, have been found to improve nucleic acid capture. Complete removal of the final wash from the lumen of the device is also needed in certain embodiments. Methods for this removal of the final wash include drying by passaging air over the surfaces of the lumen utilizing an air pump for one to three minutes. After washing, the nucleic acid is eluted from the device with a low salt buffer at higher pH than the final wash. Elution buffers are typically low ionic strength, buffered solutions at pH≧8.0, although nucleic acid can be eluted from the device with water. Elution can be carried out at ambient temperature up to about 56° C.

The design of the device permits fluids, including both liquids and gasses, to be passed through the device from one port to the other. In this way buffers can be pumped back and forth through the lumen to increase washing and elution efficiency, and air can be pumped through between washes to remove residual buffer. The device can be configured in a variety of ways with respect to introduction and removal of reagents. In one arrangement, reagents are introduced into the lumen of the device via one of the ports and removed via the other port. In a second arrangement, one port serves as both inlet and outlet for reagents, and the second port is connected to a pump that provides suction and pressure. This second arrangement avoids contacting the pump and fluid distribution control means with the reagents, and is particularly advantageous if using reagents that are corrosives or strong solvents. A third arrangement combines the first and second arrangements so that some fluids are passed completely through the device from one port to the other and other fluids are introduced and removed via the same port. For example, the nucleic acid containing sample can be introduced into the lumen via the first port and removed via the second port, and wash and elution reagents are introduced and removed via the second port using suction and air pressure applied through the first port. Those skilled in the art will recognize that many variations on these basic arrangements are possible.

As will be understood by those skilled in the art, actual working volumes will be determined by the size of the device, including lumen volume, as well as routine experimental design. For small-volume devices comparable to Pasteur pipettes, volumes will ordinarily range from about 20 μL to about 500 μL, although larger volumes up to 1 mL or as much as 2.5 mL can be used. Samples can be concentrated by reducing the volume of the elution buffer.

Quantitation of extracted nucleic acids is facilitated by the inclusion of a fluorescent compound within the elution buffer, thereby providing a rapid quality check on the extraction process while the extracted nucleic acids are still within the device. Thus, within one embodiment of the invention the nucleic acids are contacted with a fluorescent compound having a fluorescence intensity dependent on the concentration of nucleic acids, and the fluorescence of the fluorescent compound is measured. Fluorescent compounds having a fluorescence intensity dependent on the concentration of nucleic acids are fluorescent compounds that exhibit a conformation-dependent change in fluorescence intensity in the presence of nucleic acids. Useful fluorescent compounds include those compounds whose intensity increases in the presence of nucleic acids. Representative fluorescent compounds include fluorogenic minor groove binder agents such as bis-benzimide compounds and intercalating fluorogenic agents such as ethidium bromide, and commercially available fluorescent dyes (e.g., SYBR Green; Invitrogen Corp.). Fluorescent compounds can be introduced into the device in the elution buffer or immobilized in the lumen. Methods for immobilizing the fluorescent compound in the lumen and useful fluorescent compounds are described in Reed et al., U.S. Application Publication No. 20060166223 A1. The device of the invention allows for the interrogation of the lumen by fluorescence by having at least a portion of the lumen suitable for transmitting excitation energy to the fluorescent compounds in the lumen and for transmitting fluorescence emission intensity from the compounds in the lumen.

Although in principal any fluorogenic DNA-binding dye can be used in the invention, it is preferred to use a dye that is compatible with downstream processes such as PCR. A preferred dye is a bis-benzimidine (BB) dye disclosed by Reed et al., U.S. Patent Application Publication No. 20060166223 A1, which gives a strong fluorescent signal (detection at 460 nm, 40 nm filter slit width) when excited at 360 nm (40 nm slit width). The BB dye is selective for dsDNA but can also detect RNA. A popular green fluorescent dye, SYBR green (Invitrogen Corp.) is often used in so called “real time” PCR or quantitative PCR. Much like the BB dye, SYBR green can be used to both quantitate the extracted DNA before amplification and monitor the gene-specific increase during PCR. The use of fluorogenic DNA dyes or DNA probes in isothermal nucleic acid tests such as NASBA is also known.

The preferred bis-benzimidine dye, although not as sensitive as some DNA-binding dyes, has been found to be well suited for measuring genomic DNA content of a sample after extraction from DNA-rich whole blood. The minor groove-binding BB dye emits blue fluorescence in the presence of double stranded DNA, and can be added directly to PCR amplification buffer. In contrast, strong binding DNA dyes such as PICOGREEN (Invitrogen) may inhibit PCR.

Preliminary evidence indicates that the BB dye can be used in existing PCR assays if the PCR primer extension is carried out at higher annealing temperature (61.5° C. vs. 60° C.). Inclusion of the BB dye directly in the elution buffer therefore allows DNA to be measured before, during, and after gene-specific amplification. The higher primer extension temperature required with addition of BB dye may be advantageous in PCR assays (acting as a PCR enhancer). Much like the MGB TaqMan system (U.S. Pat. No. 6,727,356), A/T rich primer/target interactions are stabilized by the BB in the PCR mix, and increased duplex stability allows shorter (more specific) DNA probes to be used. The blue emitting MGB dye will likely not interfere with the green to red fluorescence wavelengths that are widely used with 2-color fluorogenic DNA probes.

RNA-selective dyes such as Ribogreen (see Molecular Probes Handbook of Fluorescent Probes and Research Products, 9th edition, Chapter 8) can also be used in the device or elution buffer. RNA-selective dyes may have advantages for real time RNA assays such as NASBA. The caveats disclosed above about inhibition of the gene-specific DNA or RNA tests also apply to RNA detecting fluorogenic dyes.

If desired, the device can be re-used following removal of residual nucleic acids and/or reagents by washing. In many cases, satisfactory washing can be achieved by running several (typically 5-10) channel volumes of distilled sterile water through the lumen. In a preferred method, the device is first washed with 5-10 channel volumes of distilled sterile water, followed by a wash with 2-3 channel volumes of 70% EtOH, which is followed by another 2-3 channel volume wash with distilled sterile water. Wash solutions can be pumped through the device using a pump (e.g., a peristaltic pump), syringe, or the like. The cleaning protocol can be carried out in through a manifold using an automated pump.

Bound nucleic acid can be stored in the device and used in later testing, including confirmation of test results. The device is rinsed with an ethanol-rich rinse and dried. Storage is at room temperature for up to several days or in a freezer for longer periods.

The invention also provides a kit comprising a nucleic acid extraction device as disclosed above and a buffer in a sealed container. The buffer can be a lysis buffer, a wash buffer, or an elution buffer as generally disclosed herein. Ordinarily, the device will be packaged with more than one buffer, commonly a complete set of buffers for extracting nucleic acid from a biological sample. For some applications, the elution buffer will comprise a fluorescent compound that exhibits a change in fluorescence intensity in the presence of nucleic acids. A typical kit comprises these components in a single package, together with a set of printed instructions for use.

The present invention has multiple applications in laboratory research, human and veterinary medicine, public health and sanitation, forensics, anthropological studies, environmental monitoring, and industry. Such applications include, without limitation, bacterial and viral detection and typing, microbial drug resistance screening, viral load assays, genotyping, infection control and pathogen screening (of, e.g., blood, tissue, food, cosmetics, water, soil, and air), pharmacogenomics, detection of cell-free DNA in plasma, white cell counting, and other fields where preparation and analysis of DNA from biological samples is of interest. As disclosed above, nucleic acids extracted using the devices and methods of the invention are readily used in a variety of downstream processes, including amplification, hybridization, blotting, and combinations thereof. The devices and methods of the invention can be employed within point-of-care diagnostic assays to identify disease pathogens, and can be utilized in genetic screening. These devices and methods can also be used within veterinary medicine for the diagnosis and treatment of animals, including livestock and companion animals such as dogs, cats, horses, cattle, sheep, goats, pigs, etc.

Nucleic acids can be extracted from a wide variety of sources. For research and medical applications, suitable sources include, without limitation, sputum, saliva, throat swabs, oral rinses, nasopharyngeal swabs, nasopharyngeal aspirates, nasal swabs, nasal washes, mucus, bronchial aspirations, bronchoalveolar lavage fluid, pleural fluid, endotracheal aspirates, cerbrospinal fluid, feces, urine, blood, plasma, serum, cord blood, blood components (e.g., platelet concentrates), blood cultures, peripheral blood mononuclear cells, peripheral blood leukocytes, plasma lysates, leukocyte lysates, buffy coat leukocytes, anal swabs, rectal swabs, vaginal swabs, endocervical swabs, semen, biopsy samples, lymphoid tissue (e.g., tonsil, lymph node), bone marrow, other tissue samples, bacterial isolates, vitreous fluid, amniotic fluid, breast milk, and cell culture supernatants. Other starting materials for extraction of nucleic acids include water samples, air samples, soil samples, cosmetics, foods and food ingredients, medical supplies and equipment, and the like.

Processes and assemblies of the present invention can be used for extraction and analysis of fragmented DNA. DNA can be fragmented by a variety of methods known in the art, such as nuclease digestion (including digestion with restriction endonucleases and DNases), sonication, heat, mechanical disruption (such as by shearing or vortexing), and chemical treatment. Applicable chemical treatments include, for example, use of metal ions such as iron (Zhang et al., Nucl. Acids Res. 29(13):e66, 2001), oxidizing agents such as bisulfite (Ehrich et al., Nucl. Acids Res. 35(5):e29, 2007), and antibiotics and drugs such as bleomycin (Chen et al., Nucl. Acids Res. 36(11):3781-3790, 2008). A preparation of fragmented DNA can contain fragments of a range of sizes or may be relatively limited in size range. Those skilled in the art will recognize that the actual size of fragments will be determined by such factors as the fragmentation method selected and the conditions used (e.g., time of treatment).

Nucleic acids prepared according to the present invention can be amplified by methods known in the art, including polymerase chain reaction (PCR) (see, e.g., Mullis, U.S. Pat. No. 4,683,202) and isothermal amplification methods. Real-time polymerase chain reaction (RT-PCR) is commonly used. See, for example, Cockerill, Arch. Pathol. Lab. Med. 127:1112-1120, 2002; and Cockerill and Uhl, “Applications and challenges of real-time PCR for the clinical microbiology laboratory,” pp. 3-27 in Reischl et al, eds., Rapid cycle real-time PCR methods and applications, Springer-Verlag, Berlin, 2002. For a review of the use of RT-PCR in clinical microbiology, see Espy et al., Clin. Microbiol. Rev. 19:165-256, 2006. Instrumentation and chemistry for carrying out PCR are commercially available. Instruments include thermal cyclers (e.g., ABI7000, 7300, 7500, 7700, and 7900, Applied Biosystems, Foster City, Calif.; LIGHTCYCLER, Roche Applied Science, Indianapolis, Ind.; SMARTCYCLER, Cepheid, Sunnyvale, Calif.; ICYCLER, Bio-Rad Laboratories, Inc., Hercules, Calif.; ROBOCYCLER and MX3000P, Stratagene, La Jolla, Calif.), detection systems for use with fluorescent probes (e.g., MYIQ and CHROMO4, Bio-Rad Laboratories, Inc.), nucleic acid analyzers (e.g., Rotor-Gene 6000, Corbett Life Science, Concorde, NSW, Australia), and amplification and detection systems (e.g., BD PROBETEC ET, Becton Dickinson, Franklin Lakes, N.J.). Other PCR technologies include fluorescent dyes for quantitative PCR (e.g., SYBR, Invitrogen Corp.) and fluorogenic probes, including FRET (fluorescent resonance energy transfer) hybridization probes (Walker, Science 296:557-559, 2002), TAQMAN probes (Applied Biosystems, Foster City, Calif.; see, Kutyavin et al., Nucl. Acids. Res. 28:655-661, 2000), ECLIPSE probes (Nanogen, Bothell Wash.), and molecular beacons (U.S. Pat. Nos. 5,925,517 and 6,150,097. Isothermal amplification methods known in the art include nucleic acid sequence-based amplification (NASBA) (Malek et al., U.S. Pat. No. 5,130,238; Compton, Nature 350:91-92, 1991; Deiman et al., Mol. Biotechnol. 20:163-179, 2002), branched DNA (Alter et al., J. Viral Hepat. 2:121-132, 1995; Erice et al., J. Clin. Microbiol. 38:2837-2845, 2000), transcription mediated amplification (Hill, Expert. Rev. Mol. Diagn. 1:445-455, 2001), strand displacement amplification (Walker, PCR Methods and Applications 3:1-6, 1993; Spargo et al., Mol. Cell Probes 10:247-256, 1996), helicase-dependent amplification (Vincent et al., EMBO Rep. 5:795-800, 2004), loop-mediated isothermal amplification (Notomi et al., Nucl. Acids Res. 28:E63, 2000), INVADER assay (Olivier et al., Nucl. Acids Res. 30:e53, 2002; Ledford et al., J. Mol. Diagn. 2:97-104, 2000), cycling probe technology (Duck et al., BioTechniques 9:142-148, 1990; Cloney et al., Mol. Cell Probes 13:191-197, 1999), rolling circle amplification (Fire and Xu, Proc. Nat. Acad. Sci. USA 92:4641-4645, 1995; Liu et al., J. Am. Chem. Soc. 118:1587-1594, 1996), and Q-beta replicase (Shah et al., J. Clin. Microbiol. 32:2718-2724, 1994; Shah et al., J. Clin. Microbiol. 33:1435-1441, 1995). For a review of isothermal amplification methods, see Gill and Ghaemi, Nucleosides Nucleotides Nucleic Acids 27:224-243, 2008.

NASBA depends on the concerted action of three enzymes to amplify target nucleic acid sequences. While able to amplify double-stranded DNA, NASBA is particularly suited for amplification of RNA. Target RNA enters the cycle by binding to a first primer, which is then extended by reverse transcriptase to form a DNA/RNA hybrid. The RNA strand is removed by the action of RNase H to yield a single-stranded cDNA. This cDNA can bind to a second primer (which includes a T7 RNA polymerase promoter sequence) and then form a double-stranded intermediate by the action of the reverse transcriptase activity. The intermediate is then copied by the action of T7 RNA polymerase into multiple single-stranded RNA copies (10-1000 copies per copy of template). These RNA copies can then enter the cycle and continue generating more copies in a self-sustained manner. Based on the NASBA mechanism, two products can be detected: a double-stranded DNA intermediate and a single-stranded RNA product.

NASBA is conveniently used with the devices of the present invention since it is isothermal (i.e. temperature cycling is not required). A denaturation step is not necessary except when a DNA target is chosen. Two considerations when running NASBA in the devices of the present invention are heat transfer and protein adsorption. The reaction temperature should be within the range of 30° C. to 50° C., usually at least 37° C., and preferably 42° C. where primer binding is more specific. Room temperature does not support NASBA, so the channel temperature must be raised efficiently or the reaction will not work. In addition, proteins such as the NASBA enzymes readily stick to glass and some organic polymeric materials, inactivating them and stopping the NASBA cycle. Two methods to address this are (1) to preadsorb the glass with a carrier such as serum albumin, or (2) to add enough serum albumin to the NASBA reaction mixture to minimize loss of enzymes.

Additional methods of nucleic acid amplification are known in the art and can be applied to DNA prepared according to the present invention. Examples of such methods include ligase chain reaction (Wu and Wallace, Genomics 4:560-569, 1989; Barany, Proc. Natl. Acad. Sci. USA 88:189-193, 1991), polymerase ligase chain reaction (Garany, PCR Methods and Applic. 1:5-16, 1991), gap ligase chain reaction (Segev, WO 90/01069), repair chain reaction (Backman et al., U.S. Pat. No. 5,792,607), and rolling circle amplification (RCA) (Lisby, Mol. Biotechnol. 12:75-99, 1999).

As will be understood by those of ordinary skill in the art, nucleic acids prepared according to the present invention can also be detected and/or analyzed without amplification using methods known in the art. Suitable methods include, without limitation, hybridization, which can be coupled to fluorescence or immunoassay, including hybridization to oligonucleotide-nanoparticle conjugates (Park et al., U.S. Pat. No. 7,169,556) and DNA barcodes (Mirkin et al., U.S. Application Publication No. 20060040286 A1); microarray technology, which can be used for expression profiling by hybridization, diagnostics, gene identification, polymorphism analysis, and nucleic acid sequencing; hybridization protection assay (Arnold et al., Clin. Chem. 35:1588-1594, 1989); dual kinetic assay (e.g., APTIMA COMBO 2 assay, Gen-Probe Incorporated); and sequencing, including microsequencing (e.g., MICROSEQ 500 16 s rDNA bacterial identification kit, Applied Biosystems). Methods of detecting polymorphisms include massively parallel shotgun sequencing (Nature 437:326-327, 2005), which can detect previously unknown features of cell-free nucleic acids such as plasma mRNA distributions and/or methylation and histone modification of plasma DNA (Fan et al., Proc. Natl. Acad. Sci. USA 105:16266-16271, 2005) Those of ordinary skill in the art will further recognize that these and other methods can be used in combination with nucleic acid amplification.

As noted above, extracted nucleic acids can be used within methods for detecting pathogens, including bacteria, viruses, fungi, and parasites. In addition, extracted nucleic acids can be analyzed to characterize drug resistance and drug sensitivity of infectious agents (e.g., methicillin or other antibiotic resistance in Staphylocccus aureus). Many such methods are known in the art, and a number of such tests have been approved by the U.S. Food and Drug Administration for human diagnostic use and are commercially available. For example, Table 1 is a list of FDA-approved tests for Chlamydia. Additional tests are listed in Table 2. Other pathogens of interest for which nucleic acid-based tests are known include bloodborne pathogens, Coccidioides immitis, Cryptococcus, Gardnerella vaginalis, Haemophilus spp., Histoplasma capsulatum, influenza virus, Mycoplasma spp., Salmonella spp., Shigella spp., and Trichomonas vaginalis. Methods for the detection of microbial contaminants, including bacteria, viruses, fungi, and parasites, in samples of foods and other products using PCR are disclosed by, for example, Romick et al., U.S. Pat. No. 6,468,743 B1. The use of PCR in testing water samples for Enterococcus species is disclosed by Frahm and Obst, J. Microbiol. Methods 52:123-131, 2003.

TABLE 1

| | | APPROVAL | |
|---|---|---|---|
| PRODUCT | COMPANY | DATE | DESCRIPTION |
| AMPLICOR CT/NG TEST FOR | ROCHE DIAGNOSTICS | Apr. 16, 2007 | http://www.fda.gov/cdrh/pdf7/k070174.pdf |
| CHLAMYDIA TRACHOMATIS | CORPORATION | | |
| GEN-PROBE APTIMA ASSAY FOR | GEN-PROBE INC. | Jan. 22, 2007 | http://www.fda.gov/cdrh/pdf6/k063451.pdf |
| CHLAMYDIA TRACHOMATIS | | | |
| APTIMA CT ASSAY ON THE TIGRIS | GEN-PROBE INC. | Oct. 13, 2006 | http://www.fda.gov/cdrh/pdf6/k061413.pdf |
| DTS SYSTEM | | | |
| COBAS AMPLICOR CT/NG TEST | ROCHE DIAGNOSTICS | Aug. 10, 2006 | http://www.fda.gov/cdrh/pdf5/k053287.pdf |
| | CORP. | | |
| GEN-PROBE APTIMA ASSAY | GEN-PROBE INC. | Jul. 25, 2006 | http://www.fda.gov/cdrh/pdf5/k053446.pdf |
| GEN-PROBE APTIMA ASSAY | GEN-PROBE INC. | Jan. 27, 2005 | http://www.fda.gov/cdrh/pdf4/k043072.pdf |
| ROCHE AMPLICOR CT/NG TEST | ROCHE MOLECULAR | Aug. 4, 1999 | http://www.fda.gov/cdrh/pdf/k973707.pdf |
| | SYSTEMS INC. | | |
| ROCHE COBAS AMPLICOR CT/NG | ROCHE MOLECULAR | Dec. 15, 1998 | http://www.fda.gov/cdrh/pdf/k973718.pdf |
| TEST | SYSTEMS INC. | | |
| ROCHE COBAS AMPLICOR | ROCHE MOLECULAR | Jun. 13, 1997 | http://www.fda.gov/cdrh/pdf/k964507.pdf |
| CHLAMYDIA TRACHOMATIS TEST | SYSTEMS INC. | | |
| GEN-PROBE AMPLIFIED CHLAMYDIA | GEN-PROBE INC. | Nov. 27, 1996 | http://www.fda.gov/cdrh/pdf/k962217.pdf |
| TRACHOMATIS ASSAY K | | | |
| LCX CHLAMYDIA TRACHOMATIS | ABBOTT | Dec. 8, 1995 | Description for K934622 available from the |
| ASSAY | LABORATORIES | | Company |

TABLE 2

| Test | References/Products |
|---|---|
| General bacterial | Dreier et al., J. Clin. Microbiol. 42: 4759-4764, 2004. |
| contamination of platelet | Mohammadi et al., J. Clin. Microbiol. 41: 4796-4798, |
| concentrates | 2003 |
| Bacillus anthracis | Bell et al., J. Clin. Microbiol. 40: 2897-2902, 2002; |
| | Oggioni et al. J. Clin. Microbiol. 40: 3956-3963, 2002; |
| | Ellerbrok et al., FEMS Microbiol. Lett. 214: 51-59, 2002. |
| Bartonella henselae | Zeaiter et al. J. Clin Microbiol. 41: 919-925, 2003. |
| Bordetella pertussis | Reischl et al., J. Clin. Microbiol. 39: 1963-1966, 2001; |
| | Anderson et al., Clin. Microbiol. Infect. 9: 746-749, |
| | 2003. |
| Borrelia burgdorferi | Makinen et al., “Genospecies-specific melting |
| | temperature of the recA PCR product for the detection of |
| | Borellia burgdorferi sensu lato and differentiation of |
| | Borrelia garinii from Borrelia afzelii and Borrelia |
| | burgdorferi sensu stricto,” pp. 139-147 in Reischl et al., |
| | eds., Rapid cycle real-time PCR methods and |
| | applications, Springer-Verlag, Berlin, 2002 |
| Borrelia garinii | Pietila et al., J. Clin. Microbiol. 38: 2756-2759, 2000. |
| Borrelia afzelii | Pietila et al., J. Clin. Microbiol. 38: 2756-2759, 2000. |
| Campylobacter | Popovic-Uroic et al., Lab Medicine 22: 533-539, 1991; |
| | Tenover, J. Clin. Microbiol. 28: 1284-1287, 1990. |
| Chlamydia | Gaydos et al., J. Clin. Microbiol. 41: 304-309, 2003; |
| | Ikeda-Dantsuji et al., J. Med. Microbiol. 54: 357-360, |
| | 2005 |
| Chlamydophila pneumoniae | Apfalter et al., J. Clin Microbiol. 41: 592-600, 2003; |
| | Tondella et al., .J. Clin Microbiol. 40: 575-583, 2002. |
| Clostridium difficile | Belanger et al., J. Clin. Microbiol. 41: 730-734, 2003. |
| Ehrlichia chaffeensis | Loftis et al., J. Clin. Microbiol. 41: 3870-3872, 2003. |
| Enterococcus Species | E. faecalis/OE PNA FISH assay, AdvanDx, Inc., |
| | Woburn, MA; see, Sloan et al., J. Clin. Microbiol. |
| | 42: 2636-2643, 2004. |
| Escherichia coli | Frahm and Obst, J. Microbiol. Methods 52: 123-131, |
| | 2003 |
| Histoplasma capsulatum | Hall et al., J. Clin. Microbiol. 30: 3003-3004, 1992. |
| Legionella pneumophila | Wellinghausen et al., “Rapid detection and |
| | simultaneious differentiation of Legionella spp. and L. pheumophila |
| | in potable water samples and respiratory |
| | specimens by LightCycler PCR,” pp. 45-57 in Reischl et |
| | al. eds., Rapid cycle real-time PCR methods and |
| | applications, Springer-Verlag, Berlin, 2002; Welti et al., |
| | Diagn. Microbiol. Infect. Dis. 45: 85-95, 2003. |
| Legionella spp. | Herpers et al., J. Clin. Microbiol. 41: 4815-4816, 2003; |
| | Reischl et al., J. Clin. Microbiol. 40: 3814-3817, 2002. |
| Listeria monocytogenes | Okwumabua et al., Res. Microbiol. 143: 183-189, 1992. |
| Mycobacterium Spp. | Hall et al., J. Clin. Microbiol. 41: 1447-1453, 2003; |
| | Lumb et al., Pathology 25: 313-315, 1993 |
| Mycobacterium | e.g., AMPLICOR MTB, Roche Molecular Diagnostics, |
| tuberculosis | Pleasanton, CA., See, e.g., Stevens et al., J. Clin. |
| | Microbiol. 40: 3986-3992, 2002; Garcia-Quintanilla et |
| | al., J. Clin. Microbiol. 40: 4646-4651, 2002; |
| | Bruijnesteijn et al., J. Clin. Microbiol. 42: 2644-2650, |
| | 2004; Sedlacek et al., J. Clin. Microbiol. 42: 3284-3287, |
| | 2004. |
| Ethambutol resistance in M. tuberculosis | Wada et al., J. Clin. Microbiol. 42: 5277-5285, 2004. |
| Isoniazid resistance in M. tuberculosis | van Doorn et al., J. Clin. Microbiol. 41: 4630-4635, |
| | 2003; |
| Rifampin resistance in M. tuberculosis | Edwards et al., J. Clin Microbiol. 39: 3350-3352, 2001; |
| | Piatek et al., Nat. Biotechnol. 16: 359-363, 1998. |
| Mycobacterum ulcerans | Rondini et al., J. Clin. Microbiol. 41: 4231-4237, 2003. |
| Mycoplasma pneumoniae | Welti et al., Diagn. Microbiol. Infect. Dis. 45: 85-95, |
| | 2003; Ursi et al., J. Microbiol. Methods 55: 149-153, |
| | 2003. |
| Neisseria gonorrhoeae | BD PROBETEC ET, Becton Dickinson, Franklin Lakes, |
| | NJ; |
| | APTIMA COMBO 2 assay, Gen-Probe Incorporated, |
| | San Diego, CA. |
| | Gaydos et al., ibid. |
| Neisseria meningitides | Guiver et al., FEMS Immunol. Med. Microbiol. 28: 173-179, |
| | 2000; Corless et al., J. Clin. Microbiol. 39: 1553-1558, |
| | 2001. |
| Penicillin resistance in N. meningitides | Stefanelli et al. J. Clin. Microbiol. 41: 4666-4670, 2003. |
| Staphylococcus aureus | S. aureus PNA FISH assay, Advandx, Inc., Woburn, MA |
| Fluoroquinolone resistance | Lapierre et al., J. Clin. Microbiol. 41: 3246-3251, 2003. |
| in S. aureus | |
| Methicillin Resistant | e.g., XPERT MRSA (Cepheid, Sunnyvale, CA); See, |
| Staphylococcus aureus | e.g., Reischl et al., J. Clin. Microbiol. 38: 2429-2433, |
| | 2000; Tan et al., J. Clin. Microbiol. 39: 4529-4531, 2002; |
| | Fang and Hedin, J. Clin. Microbiol. 41: 2894-2899, |
| | 2003; Francois et al., J. Clin. Microbiol. 41: 254-260, |
| | 2003; Ramakrishnan et al., U.S. Application Publication |
| | No. 20060057613 A1). |
| Streptococcus pneumoniae | Greiner et al., J. Clin. Microbiol. 39: 3129-3134, 2001. |
| Penicillin resistance in S. pneumoniae | Kearns et al. J. Clin. Microbiol. 40: 682-684, 2002. |
| Group A Streptococcus | Uhl et al., J. Clin. Microbiol. 41: 242-249, 2003. |
| Group B Streptococcus | CEPHEID SMART GBS ASSAY (Cepheid, Sunnyvale, |
| | CA); |
| | Bergeron et al., N. Engl. J. Med. 343: 175-179, 2000; Ke |
| | et al., “Rapid detection of group B streptoccocci using |
| | the LightCycler instrument,” pp. 107-114 in Reischl et |
| | al, eds., Rapid cycle Real-time PCR methods and |
| | applications, Springer-Verlag, Berlin, 2002. |
| Tropheryma whipplei | Fenollar et al. J. Clin. Microbiol. 40: 1119-1120, 2002. |
| Yersinia pestis | Tomaso et al., FEMS Immunol. Med. Microbiol. 38: 117-126, |
| | 2003. |
| Fluoroquinolone resistance | Lindler et al., J. Clin. Microbiol. 39: 3649-3655, 2001. |
| in Y. pestis | |

Tests for detection and diagnosis of viruses are also known in the art. Examples of such tests are shown in Table 3.

TABLE 3

| Test | References/Products |
|---|---|
| Adenovirus | Houng et al., Diagn. Microbiol. Infect. Dis. 42: 227-236, |
| | 2002; Heim et al., J. Med. Virol. 70: 228-239, 2003; Faix |
| | et al., Clin. Infect. Dis. 38: 391-397, 2004; Lankester et |
| | al., Clin. Infect. Dis. 38: 1521-1525, 2004. |
| B19 virus | Koppelman et al., Transfusion 44: 97-103, 2004. |
| BK virus | Whiley et al., J. Clin. Microbiol. 39: 4357-4361, 2001. |
| Cytomegalovirus | Machida et al., J. Clin. Microbiol. 38: 2536-2542, 2000; |
| | Nitsche et al., J. Clin. Microbiol. 38: 2734-2737, 2000; |
| | Tanaka et al., J. Med. Virol. 60: 455-462, 2000; Gault et |
| | al., J. Clin. Microbiol. 39: 772-775, 2001; Ando et al., |
| | Jpn. J. Ophthalmol. 46: 254-260, 2002; Aberle et al., J. |
| | Clin. Virol. 25 (Suppl. 1): S79-S85; Cortez et al., J. |
| | Infect. Dis. 188: 967-972, 2003; Hermann et al., J. Clin. |
| | Microbiol. 42: 1909-1914, 2004; Hall, U.S. Pat. |
| | No. 7,354,708. |
| Enterovirus | Read et al., J. Clin. Microbiol. 39: 3056-3059, 2001; |
| | Corless et al., J. Med. Virol. 67: 555-562, 2002; Kares et |
| | al., J. Clin. Virol. 29: 99-104, 2004. |
| Epstein-Barr Virus | Lo et al., Clin. Cancer Res. 7: 1856-1859, 2001; van |
| | Esser et al., Br. J. Haematol. 113: 814-821, 2001; Patel et |
| | al., J. Virol. Methods 109: 227-233, 2003; Balandraud et |
| | al., Arthritis Rheum. 48: 1223-1228, 2003; Jebbink et al., |
| | J. Mol. Diagn. 5: 15-20, 2003. |
| Hepatitis A virus | Costa-Mattioli et al., J. Viral Hepat. 9: 101-106, 2002; |
| | Rezende et al., Hepatology 38: 613-618, 2003. |
| Hepatitis B Virus | Abe et al., J. Clin. Microbiol. 37: 2899-2903, 1999; Ide |
| | et al., Am. J. Gastroenterol. 98: 2048-2051, 2003; Aliyu |
| | et al., J. Clin. Virol. 30: 191-195, 2004; Candotti et al., J. |
| | Virol. Methods 118: 39-47, 2004; |
| Hepatitis C Virus | VERSANT HCV RNA 3.0 Assay (Bayer Healthcare, |
| | Tarrytown NY), COBAS AMPLICOR HCV TEST |
| | (Roche Molecular Diagnostics); Enomoto et al., J. |
| | Gastroenterol. Hepatol. 16: 904-909, 2001; Schroter et |
| | al., J. Clin. Microbiol. 39: 765-768, 2001; Bullock et al., |
| | Clin. Chem. 48: 2147-2154, 2002; Candotti et al., ibid.; |
| | Law et al., U.S. Application Publication |
| | No. 20070207455. |
| Hepatitis D Virus | Yamashiro et al., J. Infect. Dis. 189: 1151-1157, 2004 |
| Hepatitis E Virus | Orru et al., J. Virol. Methods 118: 77-82, 2004 |
| Herpes simplex virus | Espy et al., J. Clin. Microbiol. 38: 3116-3118, 2000; |
| | Kessler et al., J. Clin, Microbiol. 38: 2638-2642, 2000; |
| | Aberle and Puchhammer-Stockl, J. Clin. Virol. |
| | 25(Suppl. 1): S79-S85, 2002; Kimura et al., J. Med. |
| | Virol. 67: 349-353, 2002. |
| Human herpes virus | Aslanukov et al., U.S. Application Publication |
| subtypes | No. 20060252032 A1. |
| HIV-1 | Ito et al., J. Clin. Microbiol. 41: 2126-2131, 2003; |
| | Palmer et al., J. Clin. Microbiol. 41: 4531-4536, 2003; |
| | Candotti et al., ibid.; Gibellini et al., J. Virol. Methods |
| | 115: 183-189, 2004; |
| HIV-2 | Schutten et al., J. Virol. Methods 88: 81-87, 2000; Ruelle |
| | et al., J. Virol. Methods 117: 67-74, 2004 |
| Human Papillomavirus | King, U.S. Application Publication No. 20080187919 |
| | A1; Hudson et al., U.S. Application Publication |
| | No. 20070111200 A1. |
| JC virus | Whiley et al., ibid. |
| Influenza Virus | van Elden et al., J. Clin. Microbiol. 39: 196-200, 2001; |
| | Smith et al., J. Clin. Virol. 28: 51-58, 2003; Boivan et al., |
| | J. Infect. Dis. 188: 578-580, 2003; Ward et al., J. Clin. |
| | Virol. 29: 179-188, 2004. |
| Metapneumovirus | Cote et al., J. Clin. Microbiol. 41: 3631-3635, 2003; |
| | Maertzdorf et al., J. Clin. Microbiol. 42: 981-986, 2004. |
| Orthopoxvirus | Espy et al., J. Clin. Microbiol. 40: 1985-1988, 2002; Sofi |
| | Ibrahim et al., J. Clin. Microbiol. 41: 3835-3839, 2003; |
| | Nitsche et al., J. Clin. Microbiol. 42: 1207-1213, 2004. |
| Parainfluenza Virus | Templeton et al., J. Clin. Microbiol. 42: 1564-1569, |
| | 2004; Templeton et al., J. Clin. Virol. 29: 320-322, 2004. |
| Respiratory Syncytial Virus | Borg et al., Eur. Respir. J. 21: 944-951, 2003; Gueudin et |
| | al., J. Virol. Methods 109: 39-45, 2003; Mentel et al., J. |
| | Med. Microbiol. 52: 893-896, 2003; Boivan et al., J. |
| | Clin. Microbiol. 42: 45-51, 2004. |
| Respiratory syncytial virus | Guedin et al., J. Virol. Methods 109: 39-45, 2003. |
| Severe acute respiratory | Poon et al., Clin. Chem. 50: 67-72, 2004; Drosten et al., |
| syndrome coronavirus | J. Clin. Microbiol. 42: 2043-2047, 2004. |
| (SARS-CoV) | |
| Varicella zoster virus | Espy et al., J. Clin. Microbiol. 38: 3187-3189, 2000; |
| | Furuta et al., J. Clin. Microbiol. 39: 2856-2859, 2001; |
| | Weidmann et al., J. Clin. Microbiol. 41: 1565-1568, |
| | 2003; Tipples et al., J. Virol. Methods 113: 113-116, |
| | 2003. |
| West Nile virus | Lanciotti et al., J. Clin. Microbiol. 38: 4066-4071, 2000 |

Examples of tests for detection and diagnosis of fungal pathogens are shown in Table 4.

TABLE 4

| Test | References/Products |
|---|---|
| Aspergillus | Loeffler et al., J. Clin. Microbiol. 40: 2240-2243, 2002; |
| | Kawazu et al., J. Clin. Microbiol. 42: 2733-2741, 2004 |
| Blastomyces dermatitidis | ACCUPROBE Blastomyces Dermatitidis Culture |
| | Identification Test, Gen-Probe Incorporated, San Diego, |
| | CA |
| Candida | Hsu et al., J. Med. Microbiol. 52: 1071-1076, 2003; |
| | Maaroufi et al., J. Clin. Microbiol. 42: 3159-3163, 2004 |
| Coccidioides | Bialek et al., J. Clin. Microbiol. 42: 778-783, 2004 |
| Conidiobolus | Imhof et al., Eur. U. Clin. Microbiol. Infect. Dis. 22: 558-560, |
| | 2003 |
| Cryptococcus | Bialek et al., Clin. Diagn. Lab. Innumol. 9: 461-469, 2002; |
| | Hsu et al., ibid. |
| Histoplasma | Imhof et al., ibid.; Martagon-Villamil et al., J. Clin. |
| | Microbiol. 41: 1295-1298, 2003 |
| Paracoccidioides | Marques et al., Mol. Genet. Genomics 271: 667-677, 2004 |
| Pneumocystis | Larsen et al., J. Clin. Microbiol. 40: 490-494, 2002; |
| | Meliani et al., J. Eukaryot. Microbiol. 50 (Suppl): 651, |
| | 2003 |
| Stachybotrys | Cruz-Perez et al., Mol. Cell. Probes 15: 129-138, 2001 |

Examples of known tests for detection and diagnosis of parasites are shown in Table 5.

TABLE 5

| Test | References |
|---|---|
| Babesia | Krause et al., J. Clin. Microbiol. 34: 2791-2794, 1996 |
| Cryptosporidium | Jiang et al., Appl. Environ. Microbiol. 71: 1135-1141, |
| | 2005 |
| Encephalitozoon | Wolk et al., J. Clin. Microbiol. 40: 3922-3928, 2002 |
| Entamoeba | Blessmann et al., J. Clin. Microbiol. 40: 4413-4417, |
| | 2002 |
| Enterocyozoon | Menotti et al., J. Infect. Dis. 187: 1469-1474, 2003 |
| Giardia | Verweij et al., J. Clin. Microbiol 42: 1220-1223, 2004 |
| Leishmania | Bossolasco et al., J. Clin. Microbiol. 41: 5080-5084, |
| | 2003Schulz et al., J. Clin. Microbiol. 41: 1529-1535, |
| | 2003. |
| Plasmodium | Lee et al., J. Clin. Microbiol. 40: 4343-4345, 2002; |
| | Farcas et al., J. Clin. Microbiol. 42: 636-638, 2004 |
| Toxoplasma | Costa et al., J. Clin. Microbiol. 38: 2929-2932, 2000; |
| | Menotti et al. J. Clin. Microbiol. 41: 5313-5316, 2003 |
| Trichomonas | Hardick et al., J. Clin. Microbiol. 41: 5619-5622, |
| | 2003 |
| Trypanosoma | Cummings and Tarleton, Mol. Biochem. Parasitol. |
| cruzi | 129: 53-59, 2003 |

DNA prepared according to the present invention can also be used in genotyping, such as in prenatal screening, prediction of disease predisposition (e.g., hypertension, osteoporosis, early onset Alzheimer's, type I diabetes, and cardiovascular disease), toxicology, drug efficacy studies, and metabolic studies. Examples include tests for celiac disease, cystic fibrosis, HLA-B27, narcolepsy, and Tay-Sachs disease (Kimball Genetics Inc., Denver, Colo.). Tests to predict drug efficacy or dosing include, for example, ACE inhibitor responder assays, screening for DNA polymorphisms in CYP2D6 & CYP2C19 genes affecting rates of drug metabolism, screening for genes affecting tamoxifen metabolism, and genetic screening for irinotecan dosing. Genotyping of single nucleotide polymorphisms (SNPs) is disclosed by Hsu et al., Clin. Chem. 47:1373-1377, 2001 using a PCR-based assay and by Bao et al., Nucl. Acids Res. 33(2):e15, 2005 using a microarray platform. SNPs may be diagnostic of complex genetic disorders, drug responses, and other genetic traits. Tests used to guide cancer treatment include tests for BRCA-1, BRCA-2, and Her-2/Neu, including expression levels thereof. Min et al. (Cancer Research 58:4581-4584, 1998) disclose methods of screening sentinel lymph nodes for expression of tumor markers by RT-PCR. Identification of other cancer markers using nucleic acid technology is under investigation. Additional genetic tests are shown in Table 6.

TABLE 6

| Test | References/Products |
|---|---|
| Alpha hemoglobin | University of Washington Medical Center, Seattle, WA |
| | (www.labmed.washington.edu) |
| α-thalassemia | University of Washington Medical Center, Seattle, WA |
| | (www.labmed.washington.edu) |
| Beta hemoglobin | University of Washington Medical Center, Seattle, WA |
| | (www.labmed.washington.edu) |
| BRCA1 & 2 | Abbaszadegan et al., Genet. Test. 1: 171-180, 1997-98; |
| | Neuhausen and Ostrander, Genet. Test. 1: 75-83, 1997 |
| COL1A1 (osteoporosis risk) | Ralston et al., PLoS Med. 3: e90, 2006. |
| Cystic fibrosis | University of Washington Medical Center, Seattle, WA |
| | (www.labmed.washington.edu); |
| | INPLEX CF test, Third Wave Technologies, Inc., |
| | Madison, WI; Accola, U.S. Pat. No. 7,312,033 |
| Factor V Leiden Mutations | Roche Molecular Diagnostics, Pleasanton, CA; Nauck et |
| | al., Clin. Biochem. 33: 213-216, 2000. |
| | INFINITI System Assay for Factor V, AutoGenomics, |
| | Inc., Carlsbad, CA |
| Factor II Mutations | Roche Molecular Diagnostics, Pleasanton, CA; Nauck et |
| | al., Clin. Biochem. 33: 213-216, 2000. |
| | INFINITI Factor II assay, AutoGenomics, Inc., |
| | Carlsbad, CA |
| Fragile X | University of Washington Medical Center, Seattle, WA |
| | (www.labmed.washington.edu) |
| Friedreich ataxia | University of Washington Medical Center, Seattle, WA |
| | (www.labmed.washington.edu) |
| Growth hormone | Kwitek et al., WO 2006/124664 |
| secretagogue receptor | |
| polymorphisms (obesity | |
| risk) | |
| hemochromatosis | Hemochromatosis DNA Test, Kimball Genetics Inc., |
| | Denver, CO. |
| Hereditary hearing loss | University of Washington Medical Center, Seattle, WA |
| | (www.labmed.washington.edu) |
| Huntington disease screen | University of Washington Medical Center, Seattle, WA |
| | (www.labmed.washington.edu) |
| Myotonic dystrophy | University of Washington Medical Center, Seattle, WA |
| | (www.labmed.washington.edu) |
| Spinla dn bulbar muscular | University of Washington Medical Center, Seattle, WA |
| atrophy | (www.labmed.washington.edu) |
| Spinal cerebellar ataxia | University of Washington Medical Center, Seattle, WA |
| | (www.labmed.washington.edu) |
| Drug metabolism genes, | INVADER UGT1A1 molecular assay (Third Wave |
| e.g., UDP | Technologies, Inc.); Dorn, U.S. Application Publication |
| glucuronosyltransferase | No. 20080032305 A1. |
| 1A1 alleles | |
| p53 mutations | see U.S. Pat. No. 5,843,654 |
| rheumatoid arthritis: | Black et al. Ann. Intern. Med. 129: 716-718, 1998; van |
| prediction of drug response | Ede et al., Arthritis Rheum. 44: 2525-2530, 2001 |
| & toxicity | |
| Warfarin sensitivity | INFINITI Warfarin Assay and INFINITI Warfarin XP |
| | Assay (AutoGenomics, Inc., Carlsbad, CA); ESENSOR |
| | Warfarin Sensitivity Test (Osmetech Molecular |
| | Diagnostics, Pasadena, CA) |
| Prediction of anti-cancer | Hayden et al., U.S. Application Publication |
| drug sensitivity | No. 20080160533 A1; Muray et al., WO 2008/082643; |
| | Semizarov et al., WO 2008/082673 |

The present invention can also be used to detect cell-free DNA in plasma. Increased concentrations of cell-free genomic DNA are symptomatic of systemic lupus erythematosus, pulmonary embolism, and malignancy. Fetal DNA in maternal plasma or serum may be used for determination of gender and rhesus status, detection of certain haemoglobinopathies, and determination of fetal HLA status for potential cord blood donation. See, for example, Reed et al., Bone Marrow Transplantation 29:527-529, 2002. Abnormally high concentrations of circulating fetal DNA have been associated with trisomy 21 in the fetus (Lo et al., Clin. Chem. 45:1747-1751, 1999) and preeclampsia (Levine et al., Am. J. Obstet. Gynecol. 190:707-713, 2004). Methods for measuring fetal DNA in maternal plasma and serum are known in the art. See, for example, Lo et al., Lancet 350:485-487, 1997 and Lo et al., Am. J. Hum. Genet. 62:768-775, 1998. A particularly valuable application is the use of fetal DNA genotyping to determine fetal Rhesus D status using maternal plasma (Muller et al., Transfusion 48: 2292-2301, 2008).

DNA prepared according to the present invention can also be used for quantitation of residual white blood cells or WBC fragments in platelet concentrates by RT-PCR. See, for example, Lee et al., Transfusion 42:87-93, 2002; Mohammadi et al., Transfusion 44:1314-1318, 2004; and Dijkstra-Tiekstra et al., Vox Sanguinis 87:250-256, 2004.

The present invention is also applicable to veterinary medicine, including disease screening and diagnosis. For example, horses imported into Australia must be tested for equine influenza by PCR. Equine influenza can be transmitted to dogs (Crawford et al., Science 310:482-485, 2005).

The invention is further illustrated by the following non-limiting examples.

### EXAMPLES

### Example 1

The feasibility of using smooth, curved glass surfaces for the purification of DNA was tested using the inner surface of a Pasteur pipette. A blood lysate was prepared by mixing 10 μl Proteinase K (10 mg/ml), 200 μl whole blood, and 200 μl of lysis buffer (6M guanidine HCl, 20 mM EDTA, 50 mM citric acid pH 6.0, 10% Tween-20, 3% Triton X-100). After 15 minutes, 200 μl of 100% ethanol was added. The lysate was then drawn up into the Pasteur pipette and allowed to sit for about 15 minutes. The lysate was then expelled. The pipette was then washed three times with Wash 1 (2M guanidine HCl, 7 mM EDTA, 17 mM citric acid pH 6.0, 33% ethanol), and four times with Wash 2 (20 mM Tris pH 7.0, 70% ethanol). Excess ethanol was dried away under vacuum for 30 minutes. Bound DNA was eluted off the glass surface in three successive elutions, each using 200 μl TE (10 mM Tris 1 mM EDTA pH 8.0). 2 μl of each eluate was quantitated using a commercially available assay (PICOGREEN assay; Invitrogen).

Purifications were performed in triplicate and compared to a device comprising flat-glass nucleic acid capture surfaces (S-channel card B0023; see Reed et al., U.S. Application Publication No. 20090215125 A1), also in triplicate. The results of the quantitation are shown in Table 7.

TABLE 7

| | | Total Yield (ng) | | |
|---|---|---|---|---|
| Device | Sample | First Elution | Second Elution | Third Elution |
| B0023 | 1 | 10.8 | 2.7 | 1.3 |
| | 2 | 8.0 | 4.1 | 3.4 |
| | 3 | 9.2 | 2.8 | 2.1 |
| Pipette | 1 | 34.3 | 26.6 | 41.5 |
| | 2 | 4.8 | 6.5 | 6.6 |
| | 3 | 22.8 | 15.8 | 19.2 |

In this experiment, the S-channel card recovered only about 9 ng of DNA from 200 μl of blood in the first elution. In contrast, the pipette isolated more DNA (samples 1 and 3). Sample 2 dropped out from the quantitation. The reason for this is unknown. Although the total surface areas of the pipette and the S-channel card were not determined, it appears that the pipette may be more efficient in purifying DNA.

To test the functionality of the isolated DNA, PCR was performed using primers for human GAPDH. PCR reactions (50-μl volume) were run in a mixture containing 10 mM Tris pH8.0, 50 mM KCl, 3 mM MgCl₂, 200 μM dNTPs, 1 μM of each primer, 0.2 unit Taq polymerase (New England Biolabs), and 5 μl undiluted sample. The primers were G3001 (GAGATCCCTCCAAAATCAAG; SEQ ID NO:1) and G3002 (CAAAGTTGTCATGGATGACC; SEQ ID NO:2). The thermocyle profile was 1 minute at 94° C., 1 minute at 54° C., and 1 minute at 72° C. for a total of 35 cycles. 7.5 μL of each reaction was mixed with 2 μl of sample buffer (New England Biolabs) and run on a 2% agarose gel in 1×TAE (40 mM Tris-acetate pH 8.3, 1 mm EDTA) and 2 μg/ml ethidium bromide. Bands were visualized under short wave UV light and photographed.

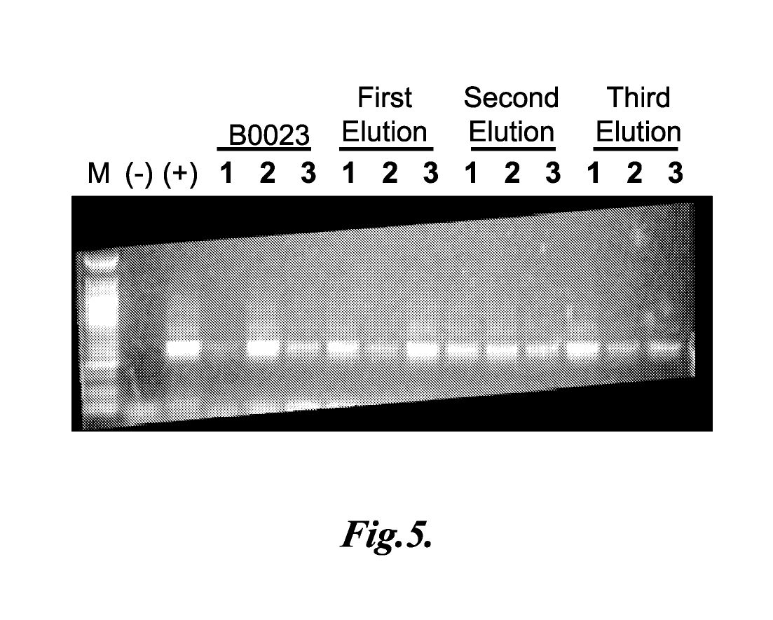

The gel analysis of the PCR products is shown in FIG. 5. The lane marked “M” contains electrophoretic mobility markers. The “(−)” and “(+)” lanes are PCR controls representing, respectively, a no-template-added control and a positive control with the addition of 10 ng of human DNA (Sigma-Aldrich). B0023 refers to S-channel purified DNA. The next nine lanes are pipette-isolated DNAs from the first, second, and third elutions. All DNAs isolated from the Pastuer pipettes were amplified very efficiently.

These results demonstrate that a smooth, curved glass surface is a suitable isolation medium for DNA from a complex biological sample (blood). DNA can be isolated in good yield and can be amplified very efficiently in PCR.

### Example 2

Glass Pasteur pipettes and glass slides (1″×3″ and 2″×3″) were compared for their ability to bind DNA. Buffers used were as disclosed in Example 1. The sample in all cases was DNA (either 500 ng or 1000 ng) in 0.6 mL binding buffer (0.2 ml Lysis Buffer+0.2 ml water+0.2 ml alcohol+DNA). DNA samples were layered onto the glass slides and allowed to sit for 30 minutes. Slides were then washed 3× with wash 1 and 6× with wash 2. Washed slides were allowed to air dry overnight. Bound DNA was eluted in three 0.2-ml aliquots of TE buffer.

For the pipettes, 0.6 mL of sample was drawn into the pipette, and the top of the pipette was sealed to hold the sample in place. The wide part of the pipette was filled to about 1.8 cm above the tapered part of the lumen up into the wider part of the lumen. The liquid was also located 6 cm into the narrow part of the lumen. After 30 minutes, the binding mixture was expelled, and the pipette was washed by drawing up into the pipette 3× wash 1, and 6× wash 2. The pipettes were allowed to air dry overnight. To elute the bound DNA, 0.2 ml TE was drawn into the pipette to rinse off the inner surface, then expelled. The pipette was allowed to drain for a bit to collect the film of TE that formed over the inner surface. The elution was repeated two more times.

Surface are of the pipette was estimated using the exterior diameter of the wide end of 0.696 cm and exterior diameter of the narrow end of 0.123 cm. Surface area was calculated from the formula: Surface area=2×Pi×radius×height (or Pi×diameter×height). For calculation purposes, half of the taper was included in the large-diameter section and half in the small-diameter section. The area covered by the liquid in the wide end of the pipette and in the narrow end were calculated and added for the total area covered by the liquid (binding mix). The calculated area was 6.2 cm², although the actual interior surface area would be expected to be somewhat less.

DNA yields were normalized to the surface area of either the slide or pipette. Results are shown in Table 8.

TABLE 8

| | Input | | | | |
|---|---|---|---|---|---|
| | DNA | Yield | Std. | Area | Ratio |
| Device | (ng) | (ng) | Dev. | (cm²) | (ng/cm²) |
| 1 × 3 | 500 | 108.4 | 9.5 | 19.4 | 5.6 |
| 2 × 3 | 500 | 291.7 | 71.2 | 38.7 | 7.5 |
| Pipette | 500 | 71.8 | 1.7 | 6.2 | 11.6 |
| 1 × 3 | 1000 | 217.2 | 6.4 | 19.4 | 11.2 |
| 2 × 3 | 1000 | 583.7 | 82.7 | 38.7 | 15.1 |
| Pipette | 1000 | 133.2 | 2.5 | 6.2 | 21.5 |

Results indicated the pipettes were about twice as effective as the glass slides in isolating DNA when normalized to the surface area. As noted above, the interior surface area of the pipette was believed to be overestimated, so the actual binding capacity was probably greater. The percent yield was lower in the pipettes, but the efficiency was higher due to the smaller surface area.

### Example 3

Twenty μL Proteinase K is mixed with 200 μL whole blood. 200 μL lysis reagent (28.7 g guanidine hydrochloride, 25 mL 0.1M sodium citrate pH 6.5, 2.5 mL 0.2M EDTA, 1 mL TRITON X-100, 3 mL TWEEN-20) is added. The solution is mixed well and incubated at 56° C. for 15 minutes. The solution is then cooled, and 200 μL ethanol is added. The contents of the tube are mixed, and the tube is centrifuged to spin down the condensate.

Using a syringe connected to one port, the entire sample is slowly loaded into the extraction device. The sample is run through the device, and the lumen is then filled with wash buffer 1 (lysis buffer without detergents diluted with equal volumes of water and 100% ethanol). The buffer is removed, and the wash is repeated. The lumen is then filled with wash buffer 2 (prepared by mixing 50 parts wash 2 concentrate (10 mL 1M Tris, 5 mL 0.5M EDTA, and 2.93 g NaCl adjusted to pH 7.4 with 5N HCl) with 30 parts water and 20 parts 100% ethanol), and the buffer is allowed to sit for 30 seconds to 12 minutes, then removed completely. This wash is repeated twice. The device is then rocked slightly back and forth to collect any adherent drops of wash 2, which are removed with a syringe.

To elute the bound DNA, 75-100 μL of TE (10 mM Tris pH 8.0, 1 mM EDTA) is loaded into the device and slowly swept through the lumen to its distal end, then back. This eluate is collected for quantitation.

### Example 4

To purify RNA from blood, commercially available buffers (Qiagen, Inc.) are utilized. Five volumes of an erythrocyte lysis solution (Buffer EL) are added to a sample of whole blood. This solution lyses red blood cells and leaves the RNA-containing white cells intact. White cells are then pelleted by centrifugation. After one additional wash to remove red cell contaminants, the white cells are lysed in buffer RLT (which contains guanidine thiocyanate). Pure ethanol is added to the lysate, which is then injected into a tubular extraction device. The device is left to stand for 20 minutes to allow the RNA to adsorb to the glass. After adsorption, the lumen is rinsed with buffer RW1 and buffer RPE (which contains ethanol). RNA is eluted from the lumen with sterile water.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A process for extracting nucleic acid from a biological sample comprising:
providing a device comprising an inner surface, an outer surface, a first port, and a second port, wherein the inner surface is composed of unmodified, smooth glass and defines a tubular lumen providing fluid communication between the first port and second port, wherein the lumen is circular, oval, or elliptical in cross-section, and wherein the lumen is essentially free of nucleic acid-specific binding sites;
introducing a nucleic acid-containing sample into the lumen of the device via one of the first and second ports;
allowing nucleic acid in the sample to bind to the unmodified smooth glass surface to produce bound nucleic acid; and
washing the bound nucleic acid.

2. The process of claim 1, further comprising eluting the bound nucleic acid from the unmodified, smooth glass surface following the washing step.

3. The process of claim 2 comprising the additional step of amplifying the eluted nucleic acid.

4. The process of claim 3 wherein the amplifying step comprises isothermal amplification.

5. The process of claim 2 wherein eluted nucleic acid is removed from the lumen via said one of the first and second ports.

6. The process of claim 2 wherein the bound nucleic acid is eluted with a buffer containing a fluorescent compound that exhibits a change in fluorescence intensity in the presence of nucleic acids.

7. The process of claim 1 wherein the lumen is a linear lumen with a longitudinal axis.

8. The process of claim 7 wherein at least a portion of the lumen is tapered along the longitudinal axis.

9. The process of claim 1 wherein the lumen is serpentine.

10. The process of claim 9 wherein the lumen is helical.

11. The process of claim 1 wherein the outer surface comprises a longitudinal ridge.

12. The process of claim 1 wherein the device comprises an inner element within the lumen, the inner element comprising an unmodified, smooth glass surface that is convex in cross-section.

13. The process of claim 1 further comprising lysing a cell sample to prepare the nucleic acid-containing sample.

14. The process of claim 1 wherein the nucleic acid-containing sample comprises a chaotropic salt.

15. The process of claim 1 wherein the nucleic acid-containing sample comprises animal nucleic acid.

16. The process of claim 15 wherein the animal nucleic acid is human nucleic acid.

17. The process of claim 1 wherein the nucleic acid is microbial nucleic acid.

18. The process of claim 1 wherein the nucleic acid is DNA.

19. The process of claim 1 wherein the nucleic acid is fragmented prior to the introducing step.

20. The process of claim 1 wherein flow of liquid through at least a portion of the lumen is turbulent.

21. The process of claim 1 wherein the washing step comprises:
introducing a wash reagent into the lumen of the device via said one of the first and second ports;
allowing the wash reagent to contact the bound nucleic acid; and
removing the wash reagent from the lumen via said one of the first and second ports.

22. An assembly comprising:
a device comprising an inner surface, an outer surface, a first port, and a second port, wherein the inner surface is composed of unmodified, smooth glass and defines a tubular lumen providing fluid communication between the first port and second port, wherein the lumen is circular, oval, or elliptical in cross-section, and wherein the lumen is essentially free of nucleic acid-specific binding sites; and
a pump in fluid communication with the lumen of the device.

23. The assembly of claim 22 wherein the pump is connected to the second port of the device.

24. The assembly of claim 23 wherein the pump is connected to the second port of the device via a manifold.

25. The assembly of claim 22 further comprising fluid distribution control means in fluid communication with the pump.

26. An assembly comprising:
a plurality of devices, wherein each device comprises an inner surface, an outer surface, a first port, and a second port, wherein the inner surface is composed of unmodified, smooth glass and defines a tubular lumen providing fluid communication between the first port and second port, wherein the lumen is circular, oval, or elliptical in cross-section, and wherein the lumen is essentially free of nucleic acid-specific binding sites;
a manifold comprising a plurality of connectors, each connecTor adapted to receive one of the devices and provide a fluid pathway into the lumen thereof via one of the ports; and
a pump in fluid communication with the manifold,
wherein each of the plurality of devices is coupled to a connector of the manifold.

27. A kit comprising:
a device comprising an inner surface, an outer surface, a first port, and a second port, wherein the inner surface is composed of unmodified, smooth glass and defines a tubular lumen providing fluid communication between the first port and second port, wherein the lumen is circular, oval, or elliptical in cross-section, and wherein the lumen is essentially free of nucleic acid-specific binding sites; and
a buffer in a sealed container, wherein the buffer is a lysis buffer, a wash buffer, or an elution buffer.

28. The kit of claim 27 wherein the buffer is an elution buffer comprising a fluorescent compound that exhibits a change in fluorescence intensity in the presence of nucleic acids.

29. The kit of claim 28 wherein the compound is a bis-benzimidine compound.
